(19) United States
(12) Patent Application Publication
(10) Pub. No.: US 2020/0179051 A1
(43) Pub. Date: Jun. 11, 2020

(54) THERAPEUTIC GUIDANCE COMPUTE NODE CONTROLLER

(71) Applicant: Vanderbilt University, Nashville, TN (US)

(72) Inventors: Michael Miga, Franklin, TN (US); Jon HEISELMAN, Nashville, TN (US); Jarrod A. COLLINS, Nashville, TN (US)

(21) Appl. No.: 16/709,455

(22) Filed: Dec. 10, 2019

Related U.S. Application Data
(60) Provisional application No. 62/777,611, filed on Dec. 10, 2018.

(51) Int. Cl. A61B 34/10; A61B 18/12; A61B 18/18; G06T 7/00; G16H 80/00; G16H 30/00

(52) U.S. Cl. CPC A61B 34/10; A61B 18/12; A61B 18/1815; A61B 2018/00577; G16H 80/00; G16H 30/00; G06T 7/0012

(57) ABSTRACT

The various examples of the present disclosure are directed towards a multi-physics controller that can predict and monitor ablation procedures. In some examples of the present disclosure, fat saturation images can be used to create custom microwave ablation bioelectric/biothermal models. In some examples of the present disclosure, a deformation correction methodology can be used. Thereby, microwave and mechanics computational models can forecast therapeutic delivery intraoperatively while correcting for deformation.

### CROSS-REFERENCE TO RELATED APPLICATIONS

This application claims the benefit of U.S. Provisional App. No. 62/777,611, filed Dec. 10, 2018, the entire contents of which are incorporated herein by reference.

### STATEMENT REGARDING FEDERALLY SPONSORED RESEARCH

This invention was made with government support under NIH Grant No. R01CA162477 awarded by the U.S. Department of Health & Human Services. The government has certain rights in the invention.

### FIELD OF THE INVENTION

The present disclosure relates to systems and methods for computationally modeling a biophysical process to guide a therapeutic device.

### BACKGROUND OF THE INVENTION

Loco-regional therapies, such as thermal ablation, have received increased indications for use in neoadjuvant roles, ablation assisted resection, and for the treatment of unresectable hepatic malignancies. While radiofrequency ablation (RFA) is the most common ablative therapy used clinically, it has presented a relatively high local recurrence rate (12-39%) when compared to microwave ablation (MWA) (6-8.8%). Additionally, in matched cohort studies, patients receiving MWA saw improved survival compared to those receiving RFA. Furthermore, MWA has received considerable interest due to its larger spatial extent of power deposition, penetration through charred tissues, and ability to ablate up to and around large vessels. Due to advances in neoadjuvant care, therapeutic options, and improved patient selection criterion, the long-term survival of patients receiving ablation treatments for hepatic colorectal cancer metastases has improved significantly in recent years, and in smaller tumors (≤3 cm) is comparable to the clinical standard of surgical resection that offers five-year survival of 44-50% in patients with metastatic colorectal cancer.

As the procedural process inherently targets internal structures, the efficacy of ablation is highly reliant on accurate localization and targeting of these subsurface diseased tissues during a procedure, as inaccurate delivery can lead to incomplete treatment and local recurrence. As such, ablations are generally performed using image-guidance (e.g. intraoperative ultrasound imaging (iUS) or computed tomography (CT)) to assist in tumor localization and probe placement. However, with these methods, real-time localization, monitoring, and assessment are extremely limited because of challenges with MR-compatibility, availability, and considerable cost.

When planning a procedure, physicians typically rely on 2D specifications of ablations from MWA device manufacturers. These 2D specifications provide expected ablation volumes given specific power and time settings. These estimates are empirically derived from ablations observed within ex vivo animal tissue. Therefore, these 2D specifications ignore the influence of patient-specific anatomical variation, tissue heterogeneity, and tissue perfusion. As a result, the manufacturer specifications are often larger and more uniform than clinically observed ablations. Additionally, using 2D specifications from animal tissues fails to integrate patient-specific data omits important variables from the procedure, such as geometric, dielectric and thermal properties of the tissue, specific heat, and the rate of blood perfusion when present. Presently, patient-specific dielectric and thermal properties are unavailable in a clinical setting, even though there is clear variation between patients with the same disease state. Therefore, a need exists for modeling frameworks that account for patient-specific variations in the state of organ tissue.

Furthermore, there is often no integration of these 2D predictions with the 3D patient images, placing a burden on the physician to mentally reconstruct and compare complex volumes when planning and performing the procedure. Such an exercise by the physician involves mentally estimating solutions to complex equations on electromagnetic wave propagation, power deposition, and biological heat transfer. Physicians cannot accurately predict many of these variables. For example, physicians often fail to account for soft-tissue deformation that occur from organ mobilization during procedures; these deformations cause substantial registration error and represent a considerable source of error for current procedures.

Computational models of MWA have attempted to solve the problems associated with coarse conventional geometric specifications used by the conventional industry by employing numerical methods to solve the differential equations governing electromagnetic (EM) wave propagation, power deposition, and biological heat transfer. For example, some methods have incorporated tissue properties that vary as a function of temperature as derived from experimental measurements or due to dynamic changes in tissue water content and blood perfusion. However, these models still neglect the variation in material properties that can occur between patients. Additionally, these methods are limited to rigid registration approaches for predicting dose distribution, which neglect soft-tissue deformations.

EM-tracking methods attempt to avoid the issues of rigid registration and accounting for intraoperative deformations by providing tracking to the real-time position of surgical tools and ultrasound (iUS) imaging. However, when compared to image-to-physical registration, the EM-iUS approach limits the subsurface information that is provided by the measurements, i.e. US data is the sole source of diseased tissue information. Additionally, EM-iUS solutions can lose efficacy when ultrasound lesion visualization is compromised, as occurs when targeting lesions in cirrhotic patients or in patients with chemotherapy-induced hyperechogenicity associated with steatosis.

Some other model-based soft-tissue deformation correction approaches have also tried to provide solutions for procedure planning, however such approaches generally have several short-comings. First, most conventional approaches fail to integrate the other complex variables of procedure planning, including patient-specific data associated with anatomical variations, tissue heterogeneity, tissue perfusion, and tissue properties (e.g. thermal, dielectric, mechanical). Second, most approaches are implemented through cumbersome add-ons to the procedure with more instruments, in order to provide the needed real-time measurements (e.g. conducting the procedure in an intraoperative magnetic resonance imaging unit to perform real-time thermometry). Third, these approaches tend to be high-cost, especially for the little additional predictive information which is provided by the approach.

Therefore, there is a need for prospective ablation modeling predictive approaches which can plan a procedure and monitor the success of the procedure in real time while taking into account patient-specific data, 3D tissue modeling, and the integration of other advanced surgical navigation methods.

### SUMMARY OF THE INVENTION

The various examples of the present disclosure are directed towards a multi-physics controller which can predict and monitor ablation procedures. In some examples of the present disclosure, fat saturation images can be used to create custom microwave ablation bioelectric/biothermal models. In some examples of the present disclosure, a deformation correction methodology can be used. Thereby, microwave and mechanics computational models can forecast therapeutic delivery intraoperatively while correcting for deformation.

A first embodiment of the present disclosure provides for a computer-implemented method. The method can provide for first obtaining spatially encoded information about patient tissues of interest during a medical procedure. The method can then provide for modeling tissue response for the patient tissues of interest associated with the medical procedure. The method can then provide for integrating the spatially encoded information and the tissue response to generate guidance during the medical procedure.

In some examples, the medical procedure can be an ablation procedure using an ablation probe.

In some examples, the tissue response can be at least one of (1) a tissue deformation to correct for a location of the ablation probe, (2) an electric field distribution generated from the ablation probe during the ablation procedure, (3) a thermal energy propagation in the patient tissues of interest due to molecular agitation from an electric field generated during the ablation procedure, or (4) an estimate of tissue damage as a result of heating during the ablation procedure. In some examples, the tissue response can be compared to spatially encoded information to guide the medical procedure. This tissue response can have been provided for by the deformation, the electric field distribution, the thermal energy propagation, or the estimate of tissue damage.

In some examples, the guidance can be at least one of (1) a location of the ablation probe or (2) a prediction of outcome for the patient tissues of interest in response to the ablation procedure and in reference to spatially encoded information.

In some examples, the ablation procedure can be a microwave ablation procedure.

In a second embodiment of the present disclosure, a system can be provided for which includes a processor and a memory. The memory can have stored therein a plurality of instructions for causing the processor to perform a method. The method can be any of the features described above with respect to the first embodiment alone, or in any combination thereof.

In some examples, the system can further include one or more instruments to collect spatially encoded information.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 shows an exemplary system for real-time monitoring and updating an ablation procedure, according to an embodiment of the present disclosure;

FIG. 2 shows an exemplary method for describing the propagation of electromagnetic waves through a target tissue, according to an embodiment of the present disclosure;

FIG. 3A shows exemplary ablation zone data as compared between a conventional method for describing electromagnetic propagation and a method according to an embodiment of the present disclosure;

FIG. 3B shows exemplary ablation zone data as compared between a conventional method for describing electromagnetic propagation and a method according to an embodiment of the present disclosure;

FIG. 3C shows exemplary ablation zone data as compared between a conventional method for describing electromagnetic propagation and a method according to an embodiment of the present disclosure;

FIG. 4 shows an exemplary method for correcting deformations, according to an embodiment of the present disclosure;

FIG. 5 shows exemplary data comparing volumetric overlap between conventional methods and a method according to the present disclosure;

FIG. 6A shows exemplary error data as compared between rigid registration methods and a method according to an embodiment of the present disclosure;

FIG. 6B shows exemplary error data as compared between rigid registration methods and a method according to an embodiment of the present disclosure;

FIG. 6C shows exemplary error data as compared between rigid registration methods and a method according to an embodiment of the present disclosure;

FIG. 7A shows exemplary ablation model predictions as compared between conventional methods and a method according to an embodiment of the present disclosure;

FIG. 7B shows exemplary ablation model predictions as compared between conventional methods and a method according to an embodiment of the present disclosure;

FIG. 7C shows exemplary ablation model predictions as compared between conventional methods and a method according to an embodiment of the present disclosure;

FIG. 7D shows exemplary ablation model predictions as compared between conventional methods and a method according to an embodiment of the present disclosure;

FIG. 7E shows exemplary ablation model predictions as compared between conventional methods and a method according to an embodiment of the present disclosure;

FIG. 7F shows exemplary ablation model predictions as compared between conventional methods and a method according to an embodiment of the present disclosure;

FIG. 8 shows an exemplary method for using fat saturation images to create custom MWA bioelectric/biothermal models, according to an embodiment of the present disclosure;

FIG. 9 shows an exemplary experimental set-up to conduct an ablation treatment, according to an embodiment of the present disclosure;

FIG. 10A shows exemplary ablation zone data as compared between rigid registration methods and a method according to an embodiment of the present disclosure;

FIG. 10B shows exemplary ablation zone data as compared between rigid registration methods and a method according to an embodiment of the present disclosure;

FIG. 10C shows exemplary ablation zone data as compared between rigid registration methods and a method according to an embodiment of the present disclosure;

FIG. 10D shows exemplary ablation zone data as compared between rigid registration methods and a method according to an embodiment of the present disclosure;

FIG. 10E shows exemplary ablation zone data as compared between rigid registration methods and a method according to an embodiment of the present disclosure;

FIG. 10F shows exemplary ablation zone data as compared between rigid registration methods and a method according to an embodiment of the present disclosure;

FIG. 11A shows exemplary temperature data according to an embodiment of the present disclosure;

FIG. 11B shows exemplary temperature data according to an embodiment of the present disclosure;

FIG. 11C shows exemplary temperature data according to an embodiment of the present disclosure;

FIG. 11D shows exemplary temperature data according to an embodiment of the present disclosure;

FIG. 11E shows exemplary temperature data according to an embodiment of the present disclosure;

FIG. 11F shows exemplary temperature data according to an embodiment of the present disclosure;

FIG. 12A shows exemplary electrical and thermal conductivity data, according to an embodiment of the present disclosure;

FIG. 12B shows exemplary electrical and thermal conductivity data, according to an embodiment of the present disclosure;

FIG. 13 shows exemplary percentage overlap between modeled and observed ablation zones, according to an embodiment of the present disclosure;

FIG. 14A shows an exemplary phantom, constructed according to an embodiment of the present disclosure;

FIG. 14B shows an exemplary T2-weighted MRI of an ablation zone, according to an embodiment of the present disclosure;

FIG. 15 shows measured fat content from MR versus actual fat for n=15 phantoms (note, a baseline was not calibrated);

FIG. 16 shows exemplary intravoxel incoherent motion imaging for perfusion and diffusion of human liver tissue;

FIG. 17 shows time lapsed video images of organ thermographic data;

FIG. 18A shows an intraoperative guidance system to be used in accordance with an embodiment of the present disclosure;

FIG. 18B shows an intraoperative guidance display for the guidance system to be used in accordance with an embodiment of the present disclosure;

FIG. 19 shows a model-based process for registration process overview, in accordance with an embodiment of the present disclosure;

FIG. 20 shows exemplary images of vascular contrast vessel extraction, in accordance with an embodiment of the present disclosure;

FIG. 21 shows a method to establish an image-driven forecasting material model in accordance with an embodiment of the present disclosure; and

FIG. 22 shows a graph with reconstructions from data with volume weighted literature values compared to nonrigid and rigid values, where the data relates to positive predictive values presented for each registered ablation as a function of average target registration of corresponding ablation antenna.

### DETAILED DESCRIPTION OF THE PREFERRED EMBODIMENTS

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents that operate in a similar manner to accomplish a similar purpose. Several preferred embodiments of the invention are described for illustrative purposes; it being understood that the invention may be embodied in other forms not specifically shown in the drawings.

The foregoing description and drawings should be considered as illustrative only of the principles of the invention. The invention is not intended to be limited by the preferred embodiment and may be implemented in a variety of ways that will be clear to one of ordinary skill in the art. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention.

The present disclosure provides a system to computationally model MWA. The system can take as input physical modeling parameters and then link the parameters with quantitative medical imaging to provide predictive procedural modeling. For example, the physical modeling parameters can include patient-specific data. This system can be integrated within the navigational environment to provide more advanced guidance in real-time during the procedure. Such a system provides clinically accurate, patient-specific computational models of MWA procedures. The present disclosure therefore provides a successful alternative to the ablation zone estimates provided by manufacturers and is also a lower-cost, less cumbersome alternative to interventional imaging strategies.

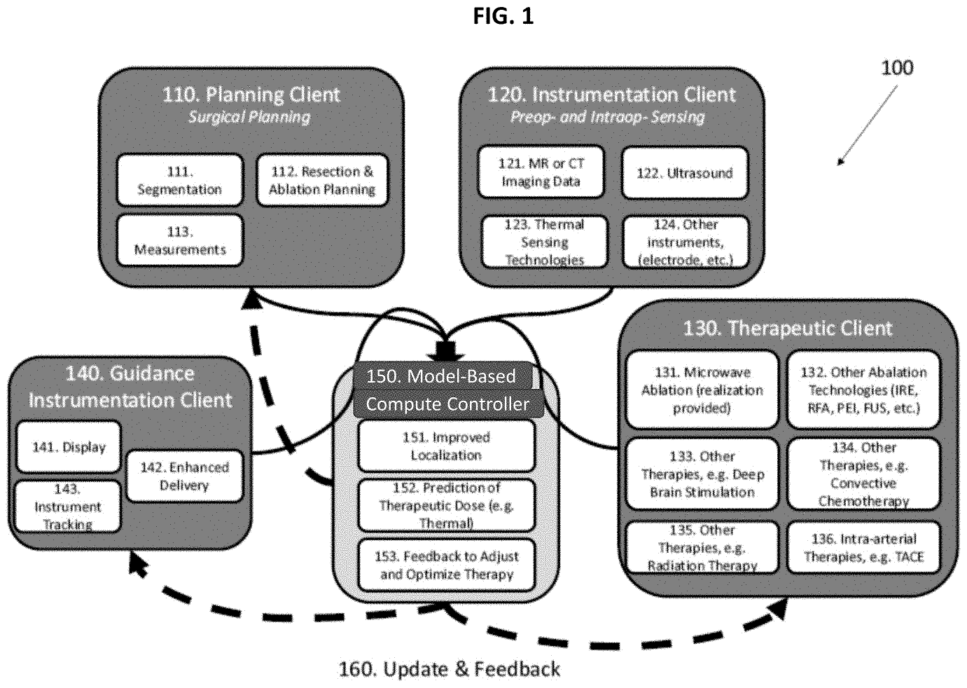

FIG. 1 demonstrates an exemplary system 100 designed to take various inputs, analyze the inputs, and plan and monitor an ablation procedure in real-time. System 100 can include a planning client 110, an instrumentation client 120, a therapeutic client 130, a guidance instrumentation client 140, a model-based compute controller 150, and an update and feedback protocol 160.

A planning client 110 can provide for the surgical planning of a procedure. Planning client 110 can provide for segmentation 111, resection planning 112, and taking measurements 113. Segmentation 111 can provide visualization of the tissue to be targeted. Resection and ablation planning 112 can determine the location of the tissue to be targeted and the trajectory of instrument delivery. It can also be used to anticipate and estimate geometric changes in the case of therapy consisting of combined resection and ablation. The planning client 110 can also provide for taking measurements of the tissue 113 as needed for the segmentation 111 and the resection and ablation planning 112. The planning client 110 can provide for additional surgical planning tools as known in the art. The planning client 110 can pass the surgical plan along to the model-based computer controller 150.

An instrumentation client 120 can provide for collecting pre-operative and intra-operative sensory data. The instrumentation client 120 can collect MR or CT imaging data 121, ultrasound data 122, thermal sensing technologies data 123, and additional instruments data 124. For example, the instrumentation client 120 can retrieve MR imaging data 121 before the procedure and ultrasound data 122 collected during the procedure. MR imaging data 121 can provide patient-specific information on target and potentially tissue properties and ultrasound data 122 can be used as limited data to assist in guidance during a procedure. For example, the MR imaging data 121 can include T1-weighted MR scans imported to create tissue geometries. MR imaging data 121 can also include (1) diffusion weighted scans imported to illuminate biomass transport (mass transport) properties, (2) fat quantification and spectroscopic magnetic resonance images imported to help establish dielectric and thermal properties, (3) magnetic resonance elastography data imported to determine mechanical stiffness of the organ of interest, (4) angiographic data to establish the vasculature of the organ. Ultrasound data 122 could be used intraoperatively to find internal structures, measure stiffness, or evaluate blood flow velocity and perfusion.

The instrumentation client 120 can also collect data from thermal sensing technologies 123 used during the procedure. For example, thermal sensing technologies 123 can determine a temperature of the ablation equipment, of the target tissue, or of tissues adjacent to the target tissue. Lastly, the instrumentation client 120 can collect measurements collected by additional instruments 124 used in the procedure. These additional instruments 124 can be any instruments as known in the art to be used to provide information for ablation or resection procedures. For example, intraoperative electrodes could be used to measure liver tissue electrical conductivity. The instrumentation can pass the collected measurements along to the model-based computer controller 150.

The therapeutic client 130 can provide data on when specific ablation procedures should be successful and what methods need to be employed for each ablation procedure. For example, the therapeutic client 130 can include data on microwave ablation 131, other ablation technologies 132, deep brain stimulation 133, convective chemotherapy 134, radiation therapy 135, and intra-arterial therapies 136. In some examples, a specific treatment can be chosen from the therapeutic client 130 and passed along to the model-based computer controller 150. In other examples, the model-based computer controller 150 can request several procedures from the therapeutic client 130 in order to compare the treatment options and see which would work best for the target tissue.

The guidance instrumentation client 140 can include a display 141, an enhanced delivery instrumentation 142, and instrument tracking sensors 143. The guidance instrumentation client 140 can therefore track the instruments used to execute the procedure in real-time. The tracking can be visually represented on the display 141. The guidance instrumentation client 140 can collect data from the enhanced delivery instrumentation 142 and any other tracking sensors 143 on surgical instruments as known in the art. The guidance instrumentation client 140 can pass the collected data along to the model-based computer controller 150.

Therefore, the model-based computer controller 150 can receive input from the planning client 110, the instrumentation client 120, the therapeutic client 130, and the guidance instrumentation client 140. The model-based computer controller 150 can use the input to provide improved localization 151, a prediction of a therapeutic dose 152, and feedback to adjust and optimize therapy 153.

For example, at 151, the input can better identify a location of the treatment instrumentation in the tissue and determine whether the treatment instrumentation is reaching the targeted tissue or if in the path to navigate target has structures that should be avoided. If the model-based computer controller 150 determines that the treatment instrumentation is not appropriately reaching the targeted tissue or is traveling a trajectory that will induce serious damage to organ structures, the model-based computer controller can send the determination to the update and feedback protocol 160.

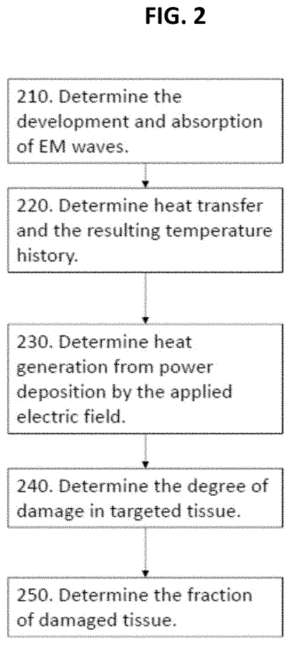

At 152, the model-based computer controller 150 can provide a prediction of the therapeutic dose, based on the therapeutic client 130. Exemplary methods of determining a therapeutic dose are discussed later with respect to method 200 of FIG. 2. The therapeutic dose can be delivered to the guidance instrumentation client 140 via the update and feedback protocol 160. In addition, planning information can be provided from the planning client 110, and patient data can be provided by instrumentation client 120, to the model-based computer controller 150 at 152 to simulate patient-specific predictions of dose before a procedure. This information can be provided back to planning client 110, at 112 to improve plans for resection, and ablation.

At 153, the model-based computer controller 150 can provide feedback to adjust and optimize therapy during the procedure. For example, the model-based computer controller 150 can receive data from the guidance instrumentation client 140 identifying whether the instruments have reached the targeted tissue. If the instruments have not reached the targeted tissue, the model-based computer controller 150 can direct send improved localization instructions from 151 along the update and feedback protocol 160. In another example, blood vessels can affect heat deposition in ablation. Optimal locations for microwave probes can be determined by pursuing repeated patient-specific models simulations taking into account vessels and the pre-procedural plan in 110 can be improved.

In other cases, the model-based compute controller 150 can receive thermal sensing data from the instrumentation client 120 which can identify whether the targeted tissue has received an appropriate dose of ablation. The model-based computer controller 150 can compare the data received from the instrumentation client 120 to the instructions received from the therapeutic client 130 to determine whether the procedure is proceeding as intended. If the procedure is not proceeding as intended, the model-based compute controller 150 can pass the data along to the therapeutic client 130 via the update and feedback protocol 160 and request updated instructions. The model-based computer controller 150 can received updated instructions from the therapeutic client 130 and pass the updates along the update and feedback protocol 160 to the guidance instrumentation client 140.

In some examples of system 100, not all elements may be used by the system when planning for or during the operation of a procedure.

Therefore, system 100 can use computer various inputs to produce organ-specific and/or subject-specific computational models for guiding therapeutic device implementations. System 100 can provide integrating novel forms of imaging data and instrumentation to produce detailed guidance information for the delivery or planning of therapy. The controller can use input data to guide mechanical deformation fields to compensate for deformations associated with shape change occurring between pre-operative imaging and intraoperative physical space. Therefore, the present disclosure can provide for creating appropriate tissue data spatial representations.

Although some instances of the present disclosure can focus on microwave ablation, system 100 can also be used for (1) compensating for deformations during soft-tissue image-guided surgery, (2) simulating electric fields for neuromodulation, epilepsy source detection, or energy deposition, (3) using thermal ablation computer models to assist in optimal placement of thermal ablation probes for the most effective coverage of a lesion and to determine therapeutic parameters, (4) guiding stent placement for aneurysms and/or predicting bypass success, and (5) simulating tumor growth for predicting outcomes of neoadjuvant chemotherapy or radiation therapy.

FIG. 2 provides an exemplary methodology 200 for optimizing therapy of a MWA ablation. Methodology 200 can be carried out in a finite element model software (such as COMSOL Multiphysics) and Matlab 2017b to simulate electromagnetic wave propagation and heat transfer. A computational controller can store and complete methodology 200 when completing a procedure.

Methodology 200 can commence at step 210 by determining the development and absorption of EM waves radiating from the antenna. Assuming no initial existing charge, this can be described by Equation 1:

(∇²+ω²μϵ_{c}) [symbol] =0 Equation 1

where ω [rad/s] is the angular frequency of the electromagnetic wave, μ[H/m] is the permeability, ϵ_{c} is the complex permittivity, [symbol] [V/m] is the electric field strength, and ∇□ is the Laplace operator. Typically, the frequency in microwave ablation is in the range of 900 MHz-2.5 GHz.

Methodology 200 can proceed to step 220 to determine heat transfer and the resulting temperature history as a result of the applied electric field. Heat transfer can be determined with Pennes' bioheat equation, shown in Equation 2:

$$ρ c \frac{\partial T}{\partial t} = ∇ \cdot k ∇ T + Q - {Q}_{p} + {Q}_{m} \qquad Equation 2$$

where ρ [kg/m³] is mass density, c [J/kg·K] is specific heat capacity, k [W/m·K] is thermal conductivity, T [K] is temperature, Q [W/m³] is heat generation due to absorbed electromagnetic energy, Q_{p [W/m}³] is heat loss due to perfusion, and Qₘ [W/m³] is metabolic heat generation. In some examples of the present disclosure, metabolic heat generation (Qₘ) and perfusion (Qₚ) can be excluded from equation 2 if they are not present.

Methodology 200 can then proceed to step 230 to determine heat generation from power deposition by the applied electric field. This can be calculated according to Equation 3:

Q=½σ∥E∥² Equation 3

where σ [S/m] is the electrical conductivity. E can be the applied electric field as determined from Equation 1.

In some examples of the present disclosure, step 230 can further include applying an electromagnetic wave transparent boundary condition to the outer edges of the modeling domain to prohibit microwave reflection. This can be demonstrated as in Equation 4a:

n̅×(∇× [symbol] )−jk [symbol] ×( [symbol] × [symbol] )=0 Equation 4a

where [symbol] is the direction normal to the boundary and k is the wave number. External boundaries can be set to a fixed temperature (such as the room temperature).

Step 230 can then include determining how the internal boundaries between the target tissue and the ablation antenna simulated saline cooling within the antenna. this can be modeled with a convective heat flux condition, as in Equation 4b:

[symbol]

·(−k∇T)=h·(T−Tₑₓₜ) Equation 4b

Altogether, steps 210-230 and Equations 1-3 capture the coupled nature of bioelectric and bioheat transport.

Methodology 200 can then proceed to step 240 in order to determine the degree of damage in the targeted tissue due to the applied electric field. Thermally-induced tissue damage is a function of both instantaneous temperature and thermal history (Equation 2). The Arrhenius damage integral can estimate protein denaturation. Protein denaturation can function as a proxy to cell death within the phantom. Therefore, the degree of damage in tissue experiencing hyperthermia can be determined from Equation 5:

$$α = {\int }_{0}^{t} A exp ( - \frac{{E}_{a}}{RT ( t )} ) dt \qquad Equation 5$$

where α is the degree of damage at a given time, A [1/s] is the frequency factor, E_{α} [J/mol] is the activation energy required to damage the tissue, R [J/mol·K] is the universal gas constant, and T(t) [K] is the temperature history of the tissue. The parameters E_{α} (2.8819×10⁵ [J/mol]) and A (1.8769×10⁴¹ [1/s]) are tissue dependent and are calibrated to maximize correspondence between thermal history and ablation zone contour fit in validation work. Therefore, step 240 can identify whether cell death in the targeted tissue is caused by the thermal dose.

Methodology 200 can then proceed to step 250 to determine the fraction of damaged tissue with Equation 6:

θ_{d}=1−e^{-α} Equation 6

In some instances of the present disclosure, a fraction of damaged tissue can be used to determine whether the procedure is operating as planned or whether the radiation emitted from the antenna needs to be changed.

Methodology 200 therefore can solve the bioelectric transport problem associated with the insertion and activation of a microwave antenna within biological tissue.

In some embodiments of methodology 200, the methodology can be adapted for radiofrequency ablation (RFA). For RFA, step 210 and equation 1 can be rewritten as equation 7:

∇·(−σ*∇V)=0 Equation 7

where σ*=σ−iωϵₒϵᵣ, ω is the frequency, ϵₒ and ϵᵣ are the absolute and relative permitivities. As opposed to microwave applications which operate in the 900 MHz to 2.5 GHz frequency for alternating current, RFA uses frequencies between 350 kHz and 500 kHz. In some examples, the complex components associated with σ* of equation 7 can be ignored. At this frequency, heating occurs due to common resistive heating. The relationship between electric field, [symbol] , and voltage V is [symbol] =−∇V. The remaining steps (220-250) of methodology 200 can then be carried out to optimize the RFA therapy.

In another example of methodology 200, the methodology can be adapted for irreversible electroporation (IRE). IRE comprises pulsing a static, very high voltage across the tissue. IRE also uses Equation 7 for step 210 and then carries out the remaining steps 220-250. IRE does not work primarily by thermal dose as the dose history is far shorter than MWA or RFA; rather, IRE has a large gradient of charge across the tissue which serves to enhance cell pore openings and allow for cell destruction. Therefore, the primary excitation for ablation occurs in Equation 6, where real conductivity is in use, and large potentials become the source of excitation.

In some examples of the present disclosure, methodology 200 and equations 1-7 can be embedded within a computational controller (such as the model-based compute controller 150 of FIG. 1) to allow the solution of equations 1-6 to enable prediction and control of microwave ablation (or RFA or IRE). The controller also incorporates data from imaging, guidance, and sensor systems to enable accurate prediction and control.

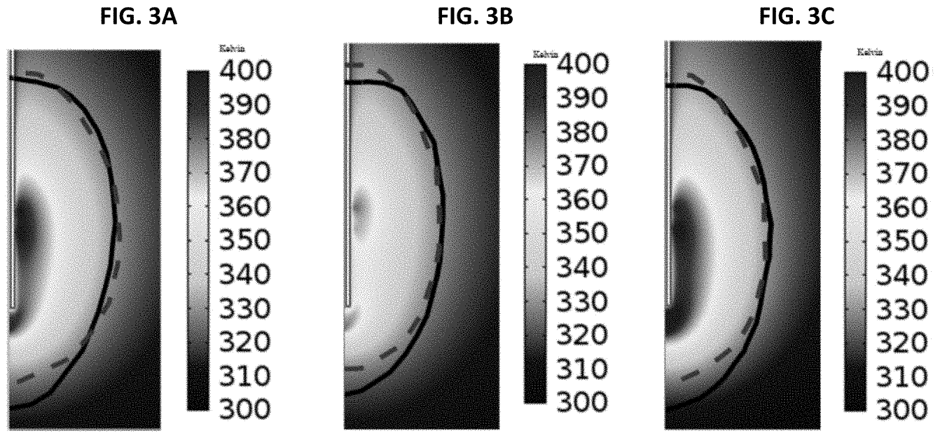

FIGS. 3A-3C show experimental data comparing a temperature map of the temperature in a target tissue as predicted by an embodiment of the present disclosure and as actually observed. The solid line demonstrates the observed ablation zone and the dashed line demonstrates the ablation zone as predicted by a model computation. The model computation shown in FIGS. 3A-3C is methodology 200 as discussed above with respect to FIG. 2. Each of FIGS. 3A-3C occurred in a different area of the target tissue with varying tissue thickness and antenna depth.

FIGS. 3A-3C directly compare the model-predicted ablation zones to a mock histology. This comparison assumes perfect registration after manually aligning the ablation antenna tip and shaft from the model space with corresponding locations in the mock histology image space. The outer ablation contours from each space were then revolved to create 3D volumes which were then compared by calculating the positive predictive value (PPV). The combined registration and modeling framework accuracy was also evaluated by computing the PPV following image-to-physical registration of the model-predicted ablation zone. Inaccuracies in both the image-to-physical registration methods and MWA modeling contribute to the error encompassed by this metric.

The computational model fitting framework is based on a nonlinear optimization approach where a parameter set defining the dielectric and thermal properties of the phantom domain within the finite element model is iteratively chosen to maximize the overlap between the model-predicted and observed ablation zones.

P=[σ, ϵ, k, c] Equation 8

where σ, ϵ, k, and c are the electrical conductivity, relative permittivity, thermal conductivity, and specific heat capacity of phantom respectively. The objective function is defined by the degree of overlap between the model-predicted and observed ablation zones as such:

$$Ω = 1 - \frac{{N}_{TP}}{{N}_{TP} + {N}_{FP} + {N}_{FN}} \qquad Equation 9$$

where Ω signifies the ratio of the intersection and union of the model-predicted and observed ablation zones. For this framework, the Nelder-Mead downhill simplex method can optimize the parameter set (equation 8) by minimizing the objective function (equation 9). The Nelder-Mead algorithm is a heuristic search approach used to solve nonlinear optimization problems without requiring derivative information.

The PPV was used to evaluate volumetric accuracy of the correction method. PPV was calculated by the following equation:

$$PPV = \frac{{N}_{TP}}{{N}_{TP} + {N}_{FP}} \qquad Equation 10$$

where NTP is the volume of the model-predicted ablation zone overlapping with the observed ablation zone and NFP is the volume of the model-predicted ablation zone which does not overlap with the true ablation zone. The PPV metric can help (1) to quantify the predictive capability of the correction method without compounding registration error and (2) to evaluate the accuracy of the combined registration and correction method framework.

The degree of ablation zone overlap for this comparison presented as the positive predictive value is, on average, 96.3±0.3%. The observed transverse and axial ablation zone dimensions gathered from mock histology were 20.1±1.0 and 31.6±1.2 mm respectively. Model-predicted ablation zone transverse and axial dimensions were 19.9±1.8 mm and 29.9±0.6 mm respectively (differing from the mock histology by 4.2% and 5.3% respectively). A t-test was used to determine significance in differences between the distributions of average TRE and PPV resulting from each evaluated registration method (α=0.05).

Therefore, FIGS. 3A-3C demonstrate the accuracy of an embodiment of the present disclosure to closely predict the ablation zone during treatment, despite different variables such as tissue thickness and antenna depth.

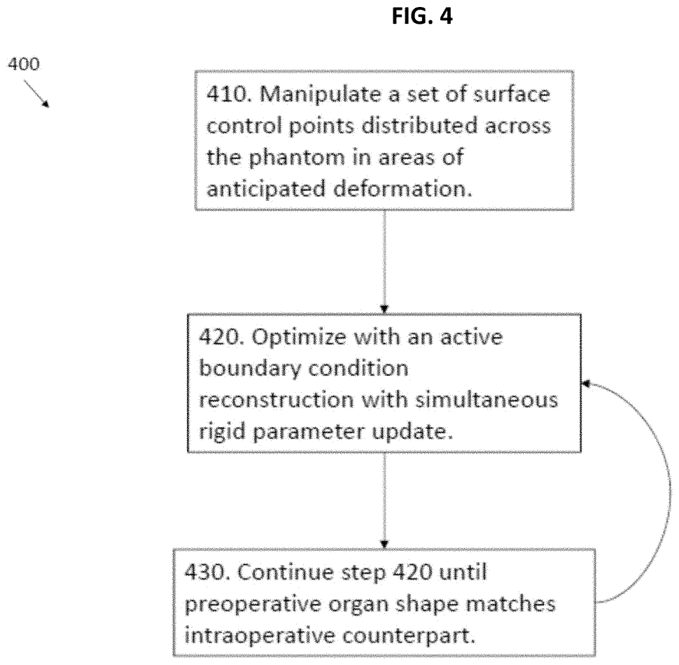

FIG. 4 shows an exemplary methodology 400 for correcting deformations, according to an embodiment of the present disclosure. Methodology 400 provides a non-rigid registration approach to correct for deformation.

Methodology 400 can commence at step 410 by manipulating a set of surface control points distributed across a liver, in areas of anticipated deformation. In an operating room, this data can be collected by a physician with an optically tracked stylus. In some examples, the data can be extracted from image volume data or conventional image-guidance instrumentation. Perturbations of the control points can provide a precomputed distribution of volumetric displacements to a biomechanical model. Data collection of the surface control points can involve the acquisition of 3D points over the anterior liver surface, and specific features (falciform, round ligament, and inferior ridges). A sparse clinical collection pattern can be taken of an anterior liver surface in its post-deformation state.

Methodology 400 can proceed to step 420 to iteratively optimize the data. The optimization process can use an active boundary condition with a simultaneous rigid parameter data. Optimization can be performed using the Levenberg-Marquardt algorithm.

At step 430, methodology 400 can provide for repeating step 420 until the preoperative organ shape matches the intraoperative counterpart.

As an example of methodology 400, surface control points of an experimental, artificially-created phantom can be manipulated to demonstrate correction for deformation. In such examples, step 410 can provide the first imaging of the mock liver using a T2-weighted MRI, from which the mock liver boundary, ablation zone, antenna tip, and antenna insertion point were segmented in the initial “pre-deformation” pose of the mock liver. Next, support blocks can be inserted beneath the mock liver to change the underlying posterior support surface shape as well as to shift the ablation to simulate an organ change from its preoperatively determined liver pose. The phantom can then be re-imaged in this “post-deformation” state, providing the same information as before but in this new deformed pose. This two-step process effectively provided all procedural delivery information and ablation physical outcomes in a mock ‘before’ and ‘after’ deforming event.

The method can proceed to step 420 which would optimize the image-to-physical registration by registering the initial pre-deformation image to the mock operating room/interventional suite (OR/IS) surface digitization of the organ generated from the post-deformation imaging data (surgical/OR setting utilizing partial surface data, interventional/IS setting utilizing full surface data). By this embodiment, the ablation information can effectively be used as a geometric target for registration assessment. In addition, a retrospective model of the MWA procedure (i.e. a model of antenna power deposition and thermal distribution) can be simulated in the pre-deformation pose given the antenna location segmented from MM. Together, these alterations to methodology 400 allow the investigation of how well a computationally-modeled, deformation-corrected ablation prediction performed versus the ground truth ablation extent and location (i.e. the evaluation of a model-based therapeutic and localization system).

Altogether, FIG. 4 provides an approach for nonrigid registration, according to an embodiment of the present disclosure. By contrast, rigid registration methods rely on the assumption that the transformation from the image to physical space is purely rigid. These methods have poor behavior when deformation is present. Methodology 400 provides a solution to accommodate for deformation.

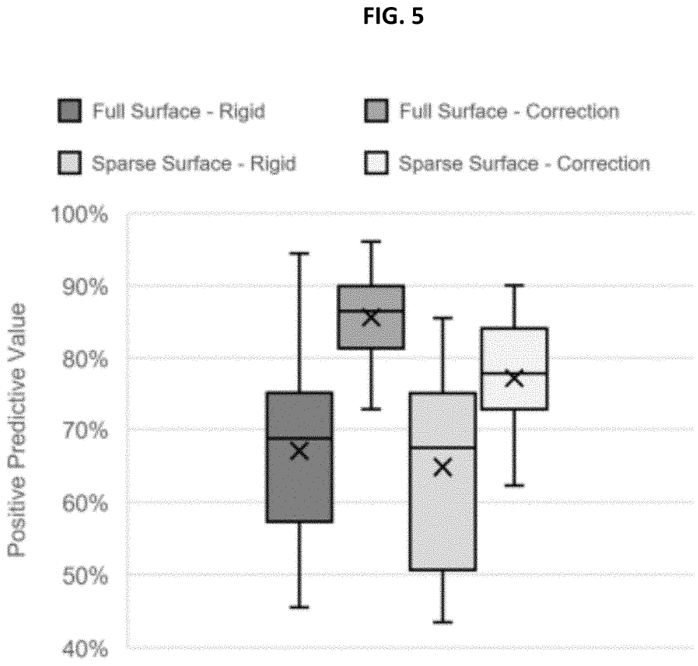

FIG. 5 shows experimental data evaluating the PPV of observed and predicted ablation zones, where the ablation zones were predicted according to method 400. The box and whiskers represent the mean, median, upper and lower quartiles, maximum, and minimum PPV for a rigid registration method of Clements et al. (Medical Physics, Vol. 35, No. 6, pp. 2528-2540, 2008) and a deformation correction method according to an embodiment of the present disclosure. FIG. 5 compares registering with full surface data and sparse surface data. FIG. 5 demonstrates that relying on a deformation correction method results in better accuracy.

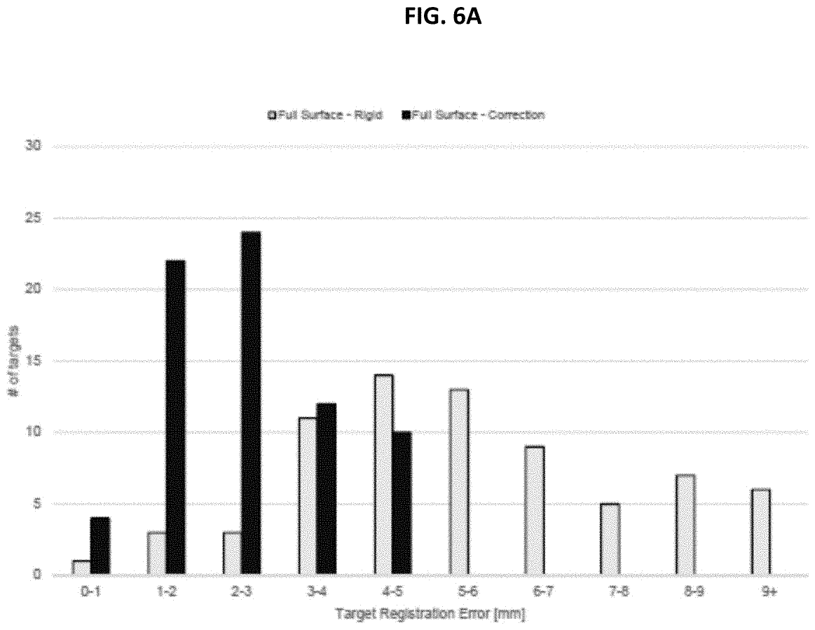

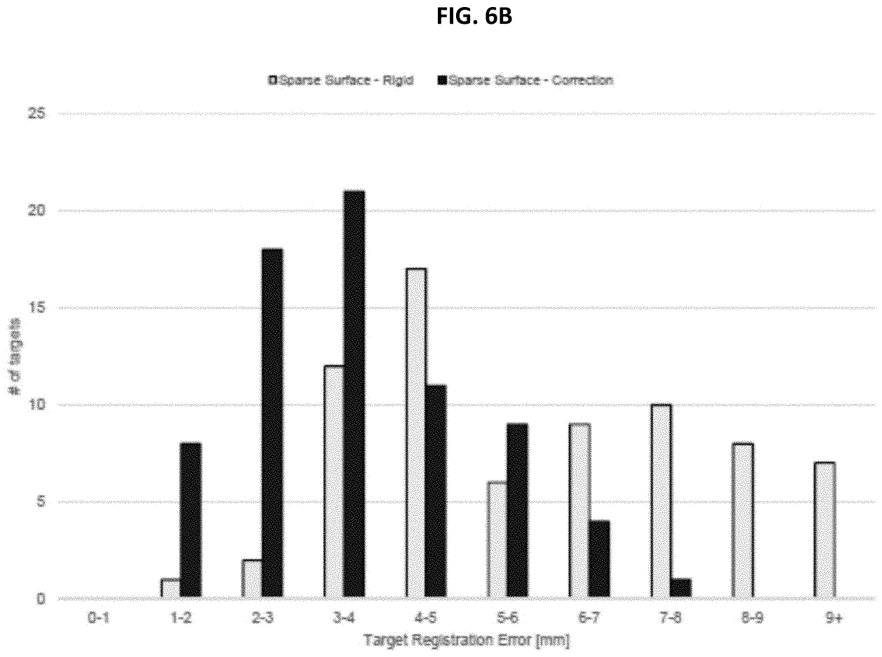

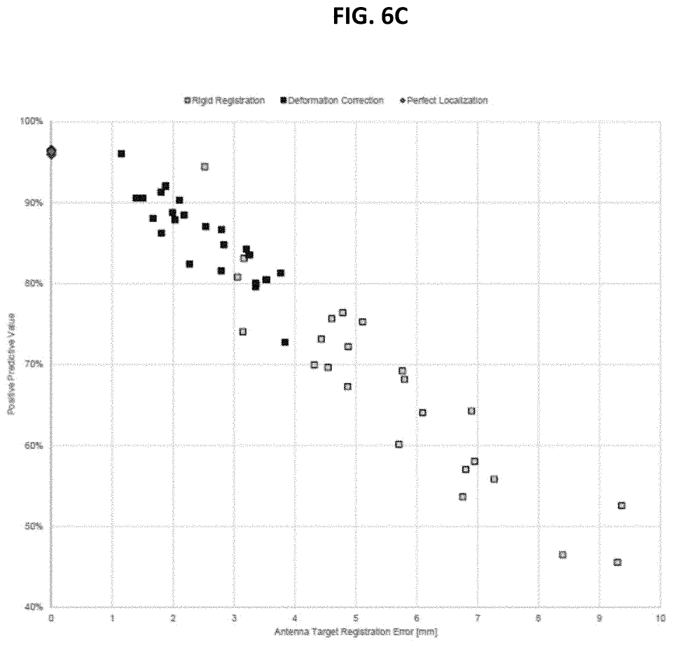

FIGS. 6A-6C show exemplary error data as compared between rigid registration methods and a correction method according to an embodiment of the present disclosure.

FIG. 6A shows exemplary target registration error data for a rigid registration method and an exemplary correction method (such as method 400 of FIG. 4A) using a full-surface image-to-physical registration approach. FIG. 6B shows target registration error data for a rigid registration method and an exemplary correction method (such as method 400 of FIG. 4A) using a sparse surface image-to-physical registration approach. FIGS. 6A-6B compare the salient feature weighted iterative closest point rigid registration method to the deformable control point nonrigid registration method. Average target registration error (TRE) is shown as the primary quantification of registration accuracy. A total of nine targets were measured across the three ablations in each image-to-physical registration scenario: (a) the antenna tip locations, (b) the antenna insertion points on the phantom surface, and (c) the centroids of the MM-segmented ablation zones. Average TRE for a registration scenario was calculated as the average distance between corresponding points in the registered image and physical data sets. FIGS. 6A-6B therefore show the accuracy of the registration methods, independent of the MWA modeling.

In addition, the quantitative results of the ablation antenna point target errors indicate that the deformation correction algorithm of represents a significant improvement over the rigid registration results (p<0.001). Furthermore, the point target errors demonstrate that a more complete source of surface data (e.g. using the full surface data often available in IS) for driving the registration provides further improvement to the deformation correction algorithm when compared to the sparse surface driving data that is available in the operating room (p<0.001). The source of surface data had much more profound impact on the deformation correction method than on the rigid registration. This observation qualitatively represents the degree to which soft-tissue deformation impacts the maximum achievable accuracy of rigid registration approaches.

FIG. 6C presents the PPV plotted as a function of the average TRE for the corresponding ablation antenna for each method of registration as well as for the perfectly localized model (i.e. TRE of 0 mm). Results depicted in FIG. 6C are from driving the registration methods with full surface data. FIG. 6C presents these results in comparison to the modeled ablation zones following registration. These novel results represent the decline in model-predictive capacity (−5.95%/mm, p<0.001, r=0.93) that is associated with localization error in a combined surgical navigation and modeling framework.

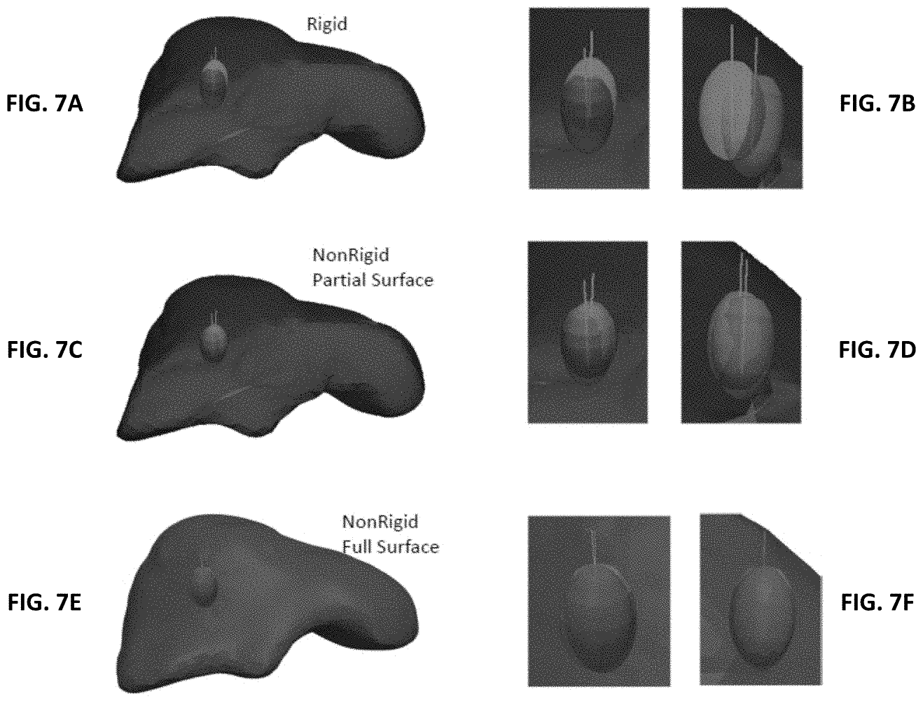

FIGS. 7A-7F show exemplary ablation model predictions as compared between conventional methods and a method according to an embodiment of the present disclosure. FIGS. 7A-7F present examples of the retrospective ablation model following rigid registration and deformation correction using sparse anterior surface data followed by complete surface data. In each figure (of FIGS. 7A-7F), the darker-colored ellipse and line represent the ground truth ablation zone and antenna pose respectively. The modeled ablation zone following rigid registration is presented in FIG. 7A and magnified in FIG. 7B. The MWA model result following deformation correction using sparse surface data, according to an embodiment of the present disclosure, is presented in FIG. 7C and magnified in FIG. 7D. The MWA model result following deformation correction using complete surface data, according to an embodiment of the present disclosure, is presented in FIG. 7E and magnified in FIG. 7F.

The example visualization of the rigid registration and deformation correction algorithms on ablation antenna positioning (of FIGS. 7A-7F) indicate that the deformation correction method provides considerable improvement to the rigid registration approach when soft-tissue deformation is present.

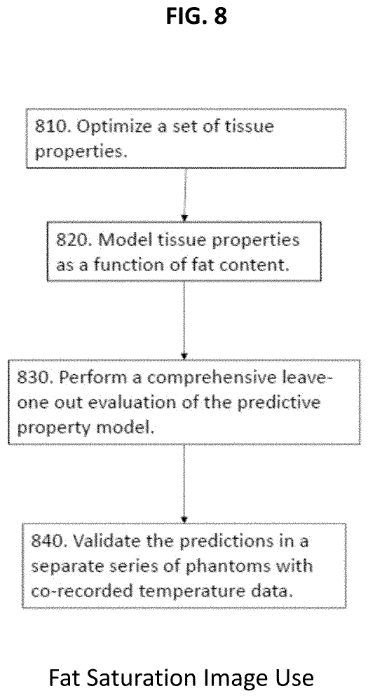

FIG. 8 shows an exemplary methodology 800 for using fat saturation images to create custom MWA bioelectric/biothermal models, according to an embodiment of the present disclosure. For example, methodology 800 can be an example of patient-specific data provided by instrumentation client 120 at 121 (as shown in FIG. 1A).

Referring back to FIG. 8, method 800 can begin at step 810 by providing for optimizing a set of tissue properties for an ablation size. In step 810, the relationship between image metrics and patient-specific model tissue properties can be established. For example, imaging sequences that resolve certain measurements of tissue that are correlated with tissue ablation processes can be determined in an optimization process. It is contemplated that the tissue ablation may be performed by radiofrequency, microwave, cryoablation, high-frequency ultrasound, or irreversible electroporation. One realization involves measuring tissue using instrumentation (for example, by the instrumentation client 120 at 121 of FIG. 1A) before and after microwave ablation. With imaging measurements performed, ablation therapy parameters known, and localization of probes known for a cohort of patients, models of each patient's ablation process can be created, and the relationship between modeled tissue properties and resulting measured ablations after therapy can be optimized. For example, fat is a tissue type that is known to have different dielectric and thermal properties than healthy liver. There is a relationship between fatty liver disease and hepatocellular carcinoma. Using methods that allow the measurement of the location of the cancer before and after ablation, imaging sequences that measure fat content within tissue, coupled with information regarding therapy settings and probe placements, a computer model can be optimized for tissue properties that enable the matching of ablation outcomes. This process of optimization can be performed for a cohort of patients and a functional relationship relating fat quantification and dielectric/thermal properties can be determined.

Method 800 can then proceed to step 820 to model tissue properties as a function of fat content. Once established, dielectric and thermal properties can be automatically determined after fat quantification pre-procedural imaging. An example of the experimental set-up embodiment of the present disclosure, step 820 can comprise constructing one (or several) phantom models with variable fat content. When multiple phantom models are created, they can each have varying fat content. Using the methods in 810, the model can then be characterized by a quantitative MR fat-imaging protocol. This can yield a predictive property model of fat content.

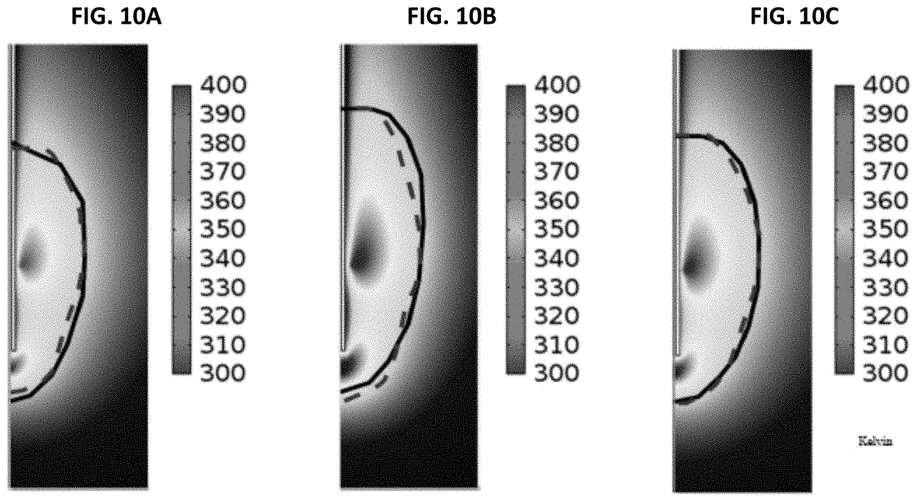

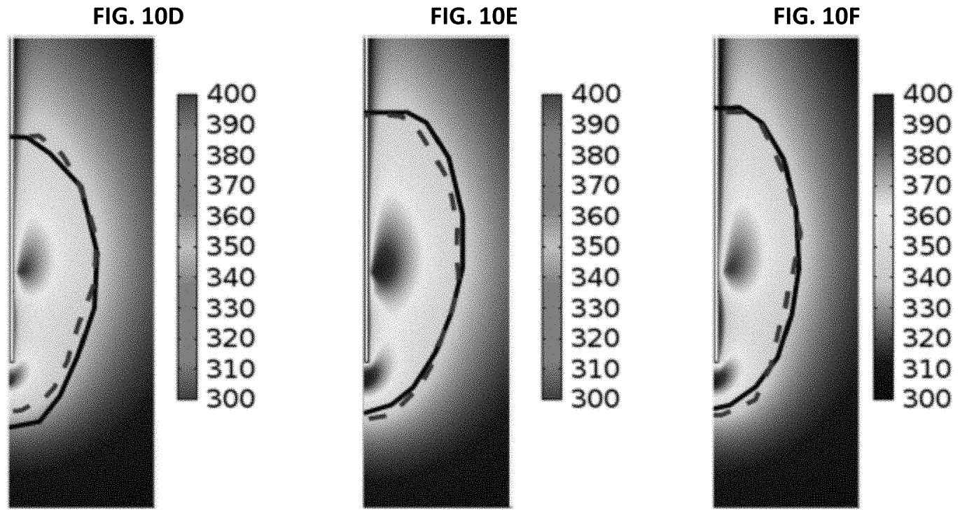

Method 800 can then proceed to step 830 to perform a comprehensive leave-one-out evaluation of the predictive property model as produced by steps 810, and 820. This leave-one-out evaluation can characterize the MR measurement-to-material property model. In some experimental set-up embodiments of the present disclosure, step 830 can be achieved by systematically holding out one phantom model constructed in step 820 from the remaining phantoms of varying fat content and using optimized property values from the remaining phantoms to create a linear regression model. The hold-out can then be used for prospective predictive testing accuracy. Cycling through each data set as a target provides some sense of predictive capability. The model-predictive accuracy can be calculated using the Jaccard similarity metric (discussed further with respect to FIGS. 10A-10F below). This can also be performed in a human cohort as demonstrated in 810.

Method 800 can complete at step 840 by validating the predictions of step 830 in a separate series of phantoms with co-recorded temperature data. While the process of 810, 820, and 830 fit a model that relates imaging properties to model properties based on matching ablation lesion sizes in patient or phantom cohorts, this represents the matching of lesion geometries. In 840, temperature sensors are used to independently measure temperature dynamics for assessing aforementioned model-building methods.

Therefore, method 800 can provide for quantitative medical imaging to estimate the dielectric and thermal properties of tissue. In some examples of method 800, organ disease states can be modeled to facilitate predictive modeling of thermal ablation.

In some examples of the present disclosure, method 800 can be combined with method 200 of FIG. 2 and method 400 of FIG. 4 to provide a modeling framework for hepatic MWA that (1) estimates tissue properties based on quantitative medical imaging of fat content, (2) better accounts for patient-specific property variation, and (3) can more accurately predict ablation outcome preoperatively. Although one MR imaging method is discussed with respect to FIG. 8, additional quantitative MR imaging methods that could complement this work (e.g. electrical conductivity imaging, elastography). In this work, we present a rigorous proof-of-concept to begin to determine the fidelity of an approach within the context of therapeutic intervention technologies.

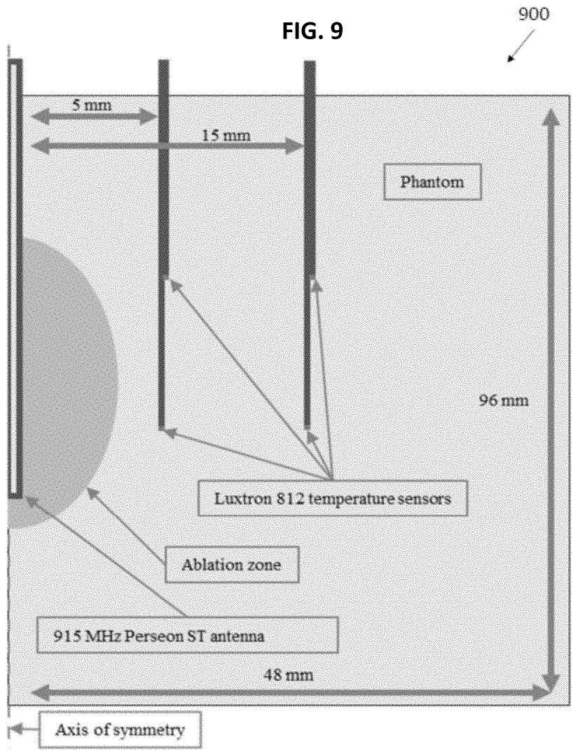

FIG. 9 shows an exemplary experimental set-up 900 to conduct an ablation treatment, according to an embodiment of the present disclosure. As depicted in FIG. 9, a 915 MHz Perseon ST microwave ablation antenna can be inserted into the center of a phantom to a recorded depth. In some examples, four temperature sensors can be used to record thermal history in each experiment of the validation phantom set with two pairs located at 5 and 15 mm transversely at varying recorded depths (observe the 4 sensor locations in FIG. 9). These locations allow for direct comparison between model-predicted and observed temperatures at a given time. The model can be discretized as a free triangular mesh with maximum element sizes of 0.15 and 2 mm for the antenna and phantom respectively. An implicit multi-frontal massively parallel sparse direct solver (MUMPS) within COMSOL Multiphysics can be used to solve both the stationary electromagnetic and transient bioheat transfer problems, as discussed above with respect to method 200.

For phantoms with no fat, two two-channel Luxtron 812 (LumaSense technologies, Santa Clara, Calif.) fiber optic temperature sensors in conjunction with 4 STB fiberoptic probes can be used to record temperatures at a rate of 2 samples per second and in the range of 0 to 120° C. The system can be accurate within ±0.5° C. and is immune to interference from radiofrequency, microwave, and electromagnetic induction.

Continuous power of 60 W can be applied for 15 minutes (for example, by the MicroThermX, Perseon Medical, Salt Lake City, Utah). Finally, a mock histology can be attained by sectioning the phantom along the midline of the MWA antenna. A 2D-representation of the ablation zone was then segmented from a photograph of the true physical ablation (an exemplary 2D-representation is shown below with respect to FIG. 14B). Measurements of the transverse and axial extents of each ablation zone can be taken from the segmented mock histology.

MRI examination of each phantom can be achieved with a 3T Intera Achieva MR scanner. Following ablation, a commercially available fat quantification sequence (such as mDixon Quant) can be used to acquire fat fraction images of each of as set of 15 phantoms for a phantom property model study (for example, according to method 800). The mDixon Quant fat quantification protocol has a reported accuracy of ±3.5% and reproducibility of ±1.4% [44]. For each phantom, 53 slices can be acquired with 3.12 mm spacing and in-plane resolution of 1.56×1.56 mm.

FIGS. 10A-10F show exemplary temperature maps of a series of 6 phantoms constructed and measured according to various embodiments of the present disclosure.

FIGS. 10A-10F show a series of 6 agar-albumin phantoms with no fat. Ablation and thermal history data were collected as described with respect to methods 200 and 400. Method 800 was then employed in an inverse fashion to determine a set of phantom electrical and thermal properties which best match the model-predicted ablation zone to the observed ablation zone. The thermal history data collected in each case were then compared to the model-predicted temperatures at those locations to validate the accuracy of the proposed phantom property reconstruction method.

Properties defining the electrical and thermal behavior of the phantom were reconstructed by deploying the MWA model within a nonlinear optimization scheme. This inverse modeling approach iteratively selected values for a parameter set, Equation 8, to maximize the overlap between the observed ablation zone and the result of the model (the result can be displayed as the Jaccard similarity coefficient, discussed further below). In total, 5 material properties (ρ, ϵ, c, σ, and k) are present within the governing equations to the model (Equations 1-3 of method 200). The conduction and electromagnetic energy deposition terms are the major contributing factors to the thermal solution of the model and are directly proportional to the thermal (k) and electrical (σ) conductivities respectively (i.e. the two properties reconstructed in the model). To reduce the dimensionality of the property reconstruction problem, density (ρ), relative permittivity (ϵ), and specific heat (c) can be assumed to remain constant with changes in fat content. Therefore, equation 8 can be rewritten below, as in equation 11:

P=[σ, k] Equation 11

In Eq. 11, σ and k are the electrical and thermal conductivities respectively. These properties were selected for our model as they directly proportional to the electrical and thermal contributions to the bioheat equation (Equations 2-3). Initial values for the parameter set, as well as other properties used in the model but not reconstructed in the optimization, can be estimated to be the average weighted linear combination of the corresponding phantom components across the phantom data set.

The degree of overlap between the observed and model-predicted ablations can be quantified by the Jaccard similarity coefficient and can be used to define the objective function in the optimization. First, binary masks representing the true and model-predicted ablation zones can be generated. Given these masked images, the number of pixels in the model-predicted ablation zone overlapping with the observed ablation zone (NTP), the number of voxels in the model-predicted ablation zone which did not overlap with the observed ablation zone (NFP), and the number of voxels in the observed ablation zone which did not overlap with the model-predicted ablation zone (NFN) can be used to calculate the similarity metric as in equation 12:

$$Jaccard = \frac{{N}_{TP}}{{N}_{TP} + {N}_{FP} + {N}_{FN}} \qquad Equation 12$$

The Jaccard similarity metric ranges from 0 (no overlap) to 1 (perfect match). Therefore, the objective function for the optimization can be as in Equation 13:

Ω=1−Jaccard Equation 13

The Nelder-Mead downhill simplex algorithm was used to optimize the parameter set for each phantom based on the minimization of the objective function in Eq. 10 [47-48]. The algorithm uses a direct search method to solve multidimensional unconstrained problems without requiring derivative information. Therefore, the approach can handle non-smooth or noisy objective functions but can take many iterations to converge. The search algorithm can be employed until a minimum first-order optimality measure of 0.01 is reached.

In FIGS. 10A-10F, the observed data is shown with the solid line and model-predicted data is shown with the dashed line. The degree of ablation zone overlap averages 93.4±2.2%, when evaluated according to the Jaccard similarity metric of equations 12-13. The average observed transverse and axial dimensions attained from mock histology following ablation were 18.2±1.4 mm and 31.0±1.2 mm respectively. Modeled ablation zone diameters differed from observed diameters by 3.3% on average while lengths differed by an average of 3.5%.

Therefore, FIGS. 10A-10F validate the inverse modeling approach to phantom property reconstruction from ablation extent data. FIGS. 10A-10F indicate a strong volumetric agreement between observed and model-predicted ablation zones in the model validation study (averaging 93.4±2.2%). These results show that the inverse modeling framework can provide accurate prediction of the margins achieved during ablation by optimizing the phantom properties for the case of the baseline phantom (i.e. no fat content). Further, as the maximization of the ablation zone overlap was employed as the objective function in the optimization, FIGS. 10A-10F also show a good model-fit correlation.

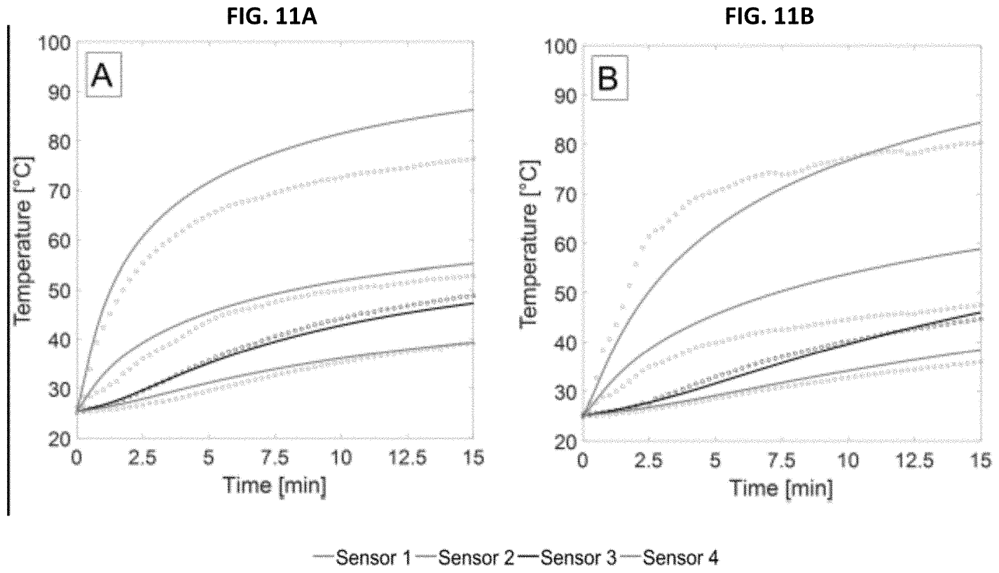

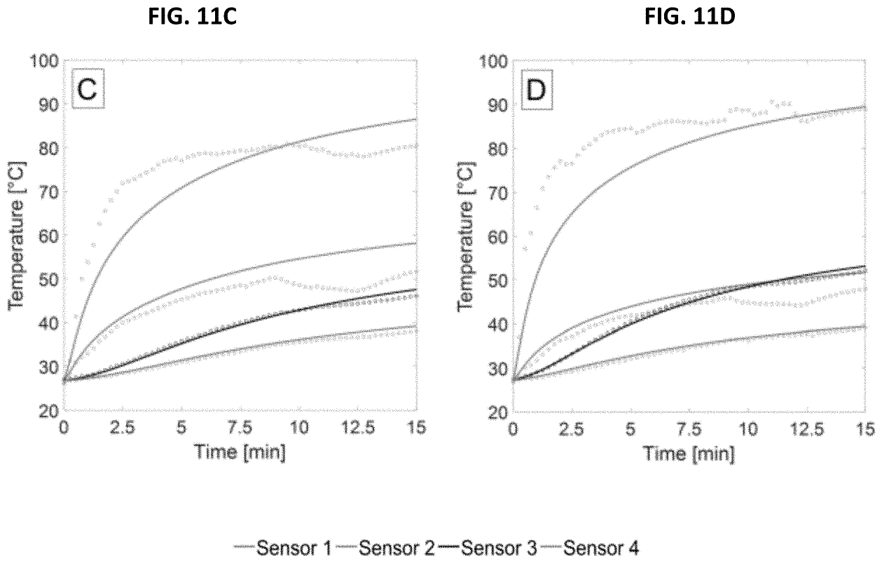

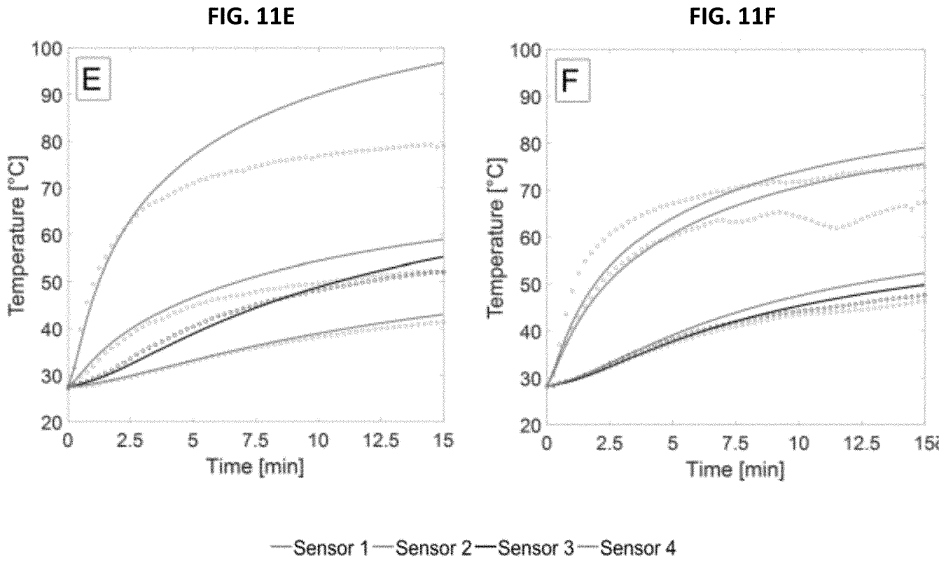

FIGS. 11A-11F show exemplary temperature data according to an embodiment of the present disclosure. FIGS. 11A-11F show thermal history observations for each temperature sensor in each phantom in an experimental set-up as shown in FIG. 9. The observed temperatures (dotted lines) are compared to model-predicted temperatures (solid lines) at those locations. The average root-mean-square (RMS) error with respect to the observed and model-predicted temperatures for each phantom averages 4.8 K.

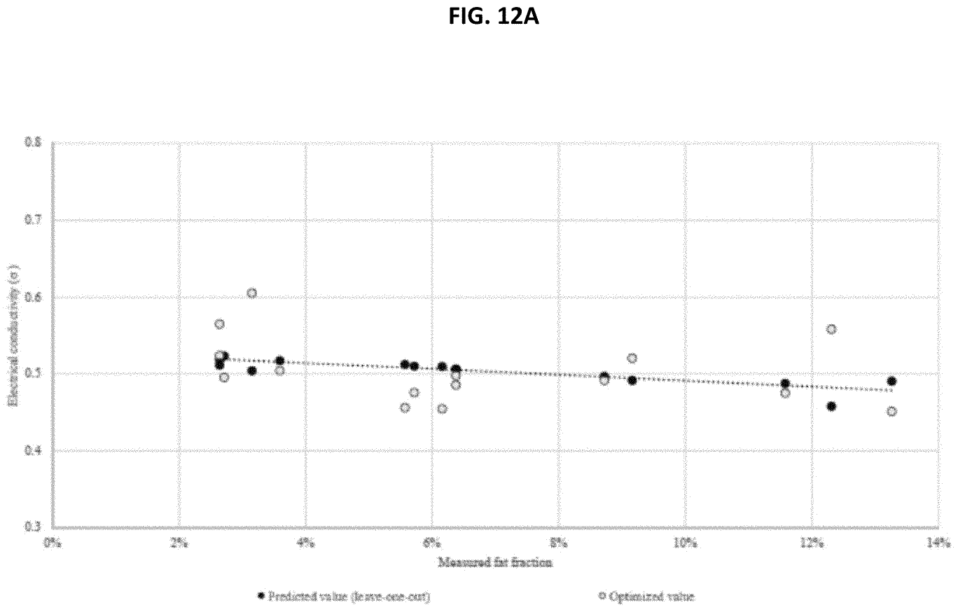

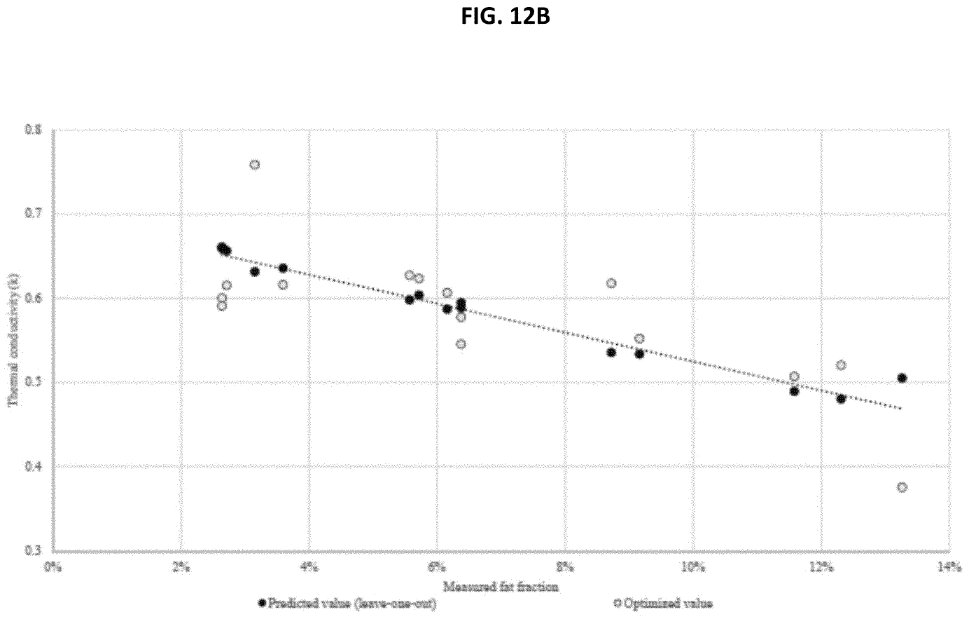

FIGS. 12A-12B show exemplary electrical and thermal conductivity data, according to an embodiment of the present disclosure. FIGS. 12A-12B show the optimized and property-model-predicted values of electrical and thermal conductivity. The dashed line represents a linear fit to the full set of 15 optimized properties as a function of measured fat content. The values of electrical (p>0.05, r=−0.32) and thermal conductivity (p<0.05, r=−0.76) are shown to decrease with fat content at rates of 0.74% and 2.61% respectively.

Results presented in FIGS. 12A-12B illustrate the correspondence between the observed and model-predicted temperatures at each of the 4 temperature sensors (average RMS error 4.8 K). As these data were purely bystander, and not utilized within the optimization, the results serve as an independent indication of the ability of the modeling framework to reconstruct phantom properties by fitting ablation zone outcomes. When considering heating near the active region of the antenna, within the zone of the largest electromagnetic energy deposition (expected to occur at the blue sensor in FIGS. 11A-11F for each case), the results show a very rapid heating within the first 5-7 minutes of the procedure which the model often under predicts before correcting by the final time point. In Equation 3 above, electromagnetic energy deposition is proportional to the value of electrical conductivity (σ). Cases C-D in FIGS. 11C-11D present the greatest rate of heating out of the 6 cases, and accordingly resulted in the reconstruction of the largest values of σ. Contrasting these results to the more gradual temperature profiles associated with the distant temperature sensors, heating far from the ablation antenna is dominated by heat transfer rather than electromagnetic energy deposition. Therefore, model-predicted temperatures throughout the procedure are more consistent (particularly for the farthest sensors from the active region of the ablation antenna).

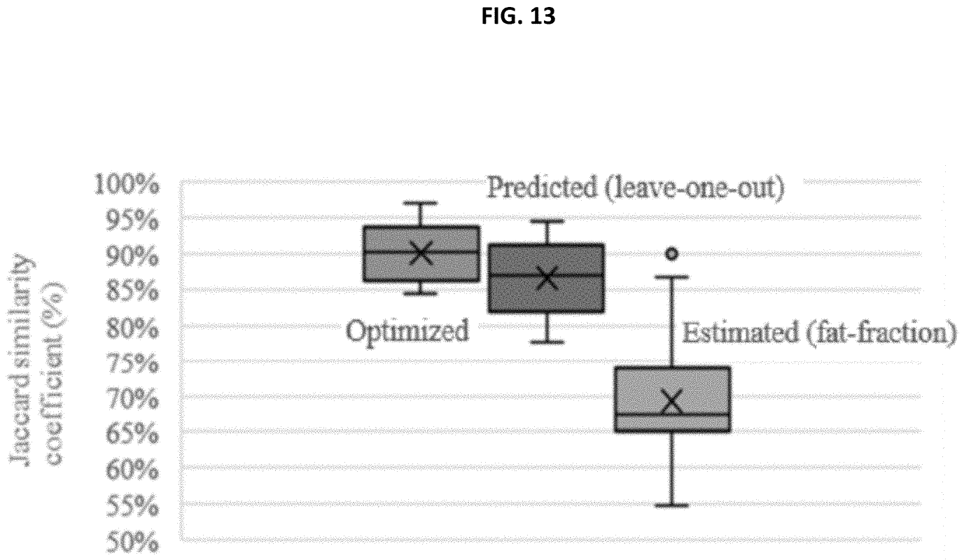

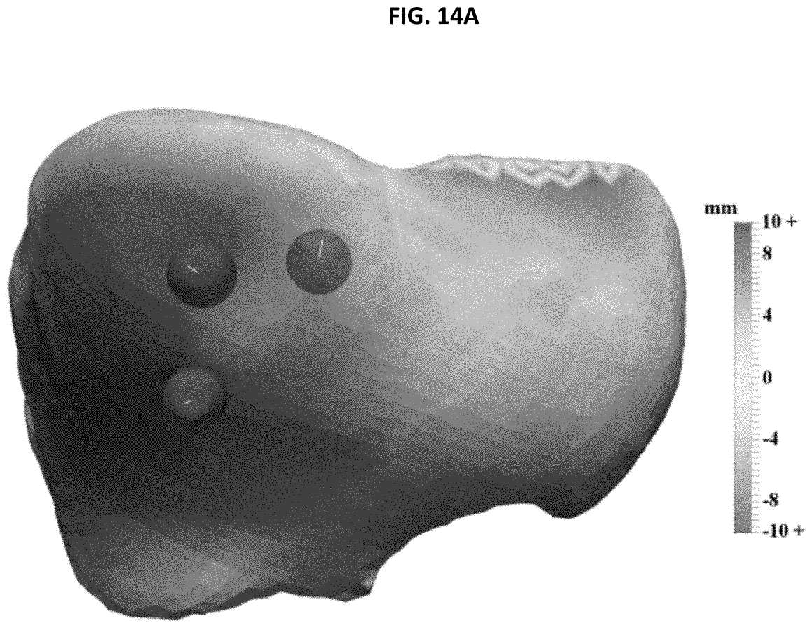

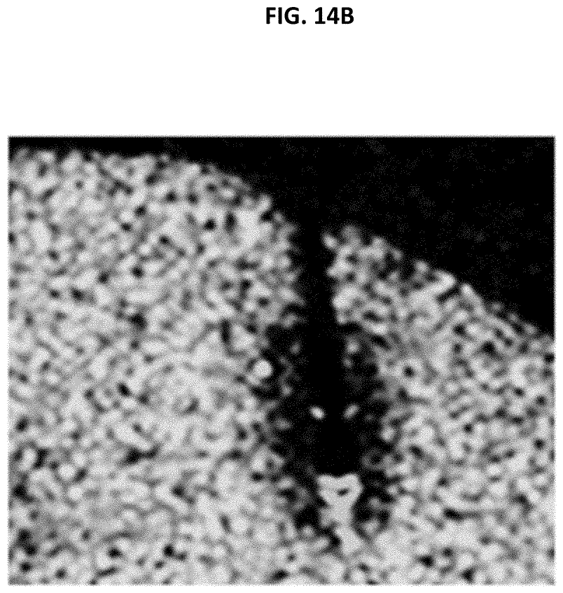

FIG. 13 shows exemplary percentage overlap between the modeled and observed ablation zones for a set of 15 agar-albumin-fat phantoms evaluated within the leave-one-out cross validation study (of method 800). The results are represented by a box and whisker chart of the distributions of the Jaccard similarity coefficient. Results are presented from using the optimized property values for each case in orange (averaging 90.2±3.8%), the predicted property values from the leave-one-out cross validation in blue (averaging 86.6±5.3%), and the estimated property values from the phantom component volume fractions (i.e. the initial guesses for the optimization) in grey (averaging 69.3±9.7%).

Altogether, FIGS. 12A-12B demonstrate phantom properties estimation based on quantitative imaging of phantom fat and matching ablation zone extent. In addition, a leave-one-out cross validation approach is provided as well for assessing fidelity. Phantom properties for each of the 15 cases were determined using the property reconstruction framework outlined in the model validation study and are represented by the light gray markers in FIGS. 12A-12B. While temperature data were not present for the phantom cases evaluated, the average ablation zone overlap following optimization (90.2±3.8%) was in accordance with the results presented in the model validation study. The leave-one-out cross validation evaluation of the proposed property model resulted in ablation zone overlap of an average 86.6±5.3%. The estimated properties for each case in this evaluation are shown with black markers in FIGS. 12A-12B. Furthermore, these results represent a 17.3% increase in ablation zone overlap when compared to a separate property model based on determining property values from the phantom component volume fractions as shown in FIG. 13.

Based on the phantom component volume fractions, it is expected that both the electrical and thermal conductivity would decrease with the addition of fat to the phantom. This expectation was realized by the property reconstructions from the inverse model solutions (FIGS. 12A-12B) which were used to construct the phantom property model. However, only the thermal conductivity is shown to have a statistically significant relationship with fat content. Additionally, using linear regression, the ablation zone areas (from mock histology) of the agar-albumin-fat phantoms is shown to significantly increase with fat content (p<0.05, r=0.86). These results clearly demonstrate that the addition of fat altered the behavior of the phantom and resulted in varying ablation extent. When considering the clinical application of these results, this could have considerable impact. As an example, because of the links between fatty liver disease and hepatocellular carcinoma, the likelihood of patient-specific variability in material properties is high and adds credence to the proposed property model framework of the present disclosure. Furthermore, the utilization of the agar-albumin-fat phantom as a surrogate for human liver tissue is adequate as the reconstructed properties are shown by the present disclosure to be within the range presented clinically.

Therefore, FIGS. 10A-13 demonstrate tissue thermal and electrical properties are an important factor in the development of therapeutic ablation zones and therefore play an important role in the accuracy and reproducibility of predictive MWA models. It is also equally apparent that these tissue properties could vary across patients and are impacted by disease state. To date, these variations in tissue properties as a function of patient-specific conditions have been excluded from approaches to predictive modeling of thermal ablation procedures with regard to current state-of-the-art therapeutic applications.

Additionally, the inverse modeling framework of method 800 can optimize a set of tissue properties that best fit the model-predicted and observed ablation zone contours in a series of phantoms of varying fat content. Therefore, the present disclosure further provides accurate model-predicted temperatures and ablation zones based on image-driven determination of tissue properties. The present disclosure also enables model-based deformation correction methods to be applied within image-guided microwave ablation procedures. This improves the accuracy and efficiency of intraoperative tumor localization and antenna placement and potentially the spatial extent of thermal dose.

### Deformation Correction Algorithm

An isotropic linearly elastic finite element model is employed to simulate deformation of the liver. At static equilibrium, linear elasticity is governed by the Navier-Cauchy equations in three dimensions:

$$\frac{E}{2 ( 1 - v )} {∇}^{2} u + \frac{E}{2 ( 1 + v ) ( 1 - 2 v )} ∇ ( ∇ \cdot u ) - F = 0 \qquad Equation 14$$

where E is the Young modulus, v is the Poisson ratio, u is displacement, and F is applied force. In this work the values E=2100 kPa and ν=0.45 are used. With the Galerkin weighted residual method on linear Lagrange basis functions, this system of partial differential equations can be rewritten as:

Ku=f Equation 15

where K is the global stiffness matrix and f is a vector of known forcing conditions. In the forward boundary value problem, the displacement vectors u throughout the domain can be solved only if displacements and forces on the boundary are known. The inverse boundary reconstruction problem attempts to resolve the distributed loading conditions on the boundary that establish the displacement response ũ that best approximates the partially observable true displacement field u without exact spatial correspondence being known.

By the principle of superposition, a basis of displacements can be constructed such that ũ=J*ᵤα*, where the matrix J*ᵤ represents displacement responses to independent unit displacements of every boundary node in each spatial direction and the vector α* represents the weight for each basis with length triple the number of boundary nodes. Solving for α* represents the full resolution boundary reconstruction problem where every boundary node on the mesh is permitted independent degrees of freedom. However, reconstructing the full resolution problem is not feasible due to computational time constraints and limited information rendering the solution extremely underdetermined.

Dimensionality can be substantially reduced by pruning the reconstructive degrees of freedom to displacements on a subset of control points distributed across the boundary. In that way, the unknown distributed load applied to the domain is approximated as a statically equivalent linear combination of responses to locally consolidated point loads. By the Saint-Venant principle, differences between the deformation responses of the true and the approximated loading configurations quickly vanish with distance. To simulate the basis of localized boundary load responses, control points are evenly spaced on the surface of the mesh using k-means clustering, with k=45. The control points are independently perturbed in each Cartesian direction to generate 3 k total modes of deformation. Displacement responses for each mode are solved from Equation 15 by applying a boundary condition with 5 mm displacement in the direction of the active control point perturbation and zero displacement boundary conditions at all remaining control points. With each resulting displacement solution, stress and strain are computed from conventional stress-strain and strain-displacement relations for linear elasticity. By focusing the total boundary condition effect from a local neighborhood into a single point, local artifact arises from approximating a smoothly varying distributed load on the surface as a series of point effects. To address these irregularities, point load responses are relaxed back onto to the boundary nodes in the local neighborhood of the control point.

To accomplish relaxation, the Saint-Venant principle is invoked again to determine a statically equivalent load that is redistributed across the locally aggregated boundary region surrounding the control point. Each point load is relaxed by establishing a radius of half the distance between control points, or equivalently the Voronoi tile of the k-means cluster, and solving Equation 15 for the self-equilibrated response of the local region when the far field displacement responses of all other boundary nodes are immobilized. Each relaxed control point response becomes a mode of variation in the reconstructive basis, representing a spatially local deformation applied to the mesh. For the purpose of reconstruction from sparse data, this approach is beneficial in comparison to more spatially distributed spectral and polynomial function bases that can produce extrapolative error when fitting local data.

With these relaxed responses to control point boundary perturbations, a displacement response matrix Jᵤ is constructed where each column of length 3M corresponds to a relaxed displacement solution vector to one of the 3 k rows of control point perturbations, where M is the number of nodes in the mesh. The relaxed stress and strain solutions for each perturbation response are similarly assembled into the stress response matrix J_{σ} and the strain response matrix J_{ϵ}. With superposition, a reconstructed deformation state that also satisfies Equation 14 can be linearized as:

u=Juα Equation 16

\tilde{σ} =J_{σα} Equation 17

\tilde{ϵ} =J_{ϵα} Equation 18

where ũ, \tilde{σ} , and \tilde{ϵ} are the approximated displacement, stress, and strain values for the deformation defined by the relaxed control point response matrices Jᵤ, J_{σ}, and J_{ϵ}, and the state vector α of length 3 k.

To solve for the deformation state, Levenberg-Marquardt optimization is employed to iteratively minimize model-data error and the strain energy of the system in a scheme that also optimizes rigid transformation parameters. This optimization of rigid parameters allows a global minimization of element rotations to reduce incurred rotational inaccuracies inherent to linear elasticity. While co-rotational models are sometimes used to compensate, these formulations incorporate geometric nonlinearities that disrupt the superposition leveraged in this application. Hence, the total deformation state to reconstruct is the parameter vector β of length 3 k+6 defined as

β=[α, τ, θ] Equation 19

where τ is a vector of rigid translations and θ is a vector of rigid rotations about the x, y, and z axes. These parameters are determined by minimizing the least squares objective function:

$$Ω ( β ) = {\sum }_{F} \frac{{w}_{F}}{{N}_{F}} {\sum }_{i = 1}^{{N}_{F}} {f}_{i}^{2} + {w}_{E} {f}_{E}^{2} \qquad Equation 20$$

where fᵢ denotes the error between the deformed model and the data point i of N_{F} total points within an intraoperatively collected point cloud for feature F, W_{F} is the weight of the feature, f_{E} is the average strain energy of the deformation state, and w_{E} is a regularizing strain energy weight that controls the deformability of the registration. This objective function distinguishes the error terms for distinct types of features that can comprise the intraoperative data. From digitization of the organ surface, features include the falciform ligament, the left and right inferior ridges, and the general anterior liver surface. From tracked iUS, features can consist of the posterior liver surface, hepatic and portal vein contours, and hepatic and portal vein centerlines. Finally, singular fiducial points can be used when they are available in controlled phantom environments that have embedded and measurable intraoperative target positions. To determine model-data error, a distance vector pᵢ is defined as:

pᵢ=yᵢ−Sᵢ (R(x₀−x̅₀+Jᵤα)+τ+x̅₀) Equation 21

where yᵢ is the intraoperative data point, x₀ are the initial coordinates of the undeformed mesh, x̅₀ is the centroid of x₀, Sᵢ is a sampling operation encoding correspondence between model and data, and the rotation matrix R is defined as:

R =R(θ)=R(θₓ)R(θ_{y})R(θ_{z}) Equation 22

The sampling operation Sᵢ is implemented as a closest point operator that selects the nearest feature point in the deformed model to yᵢ. The sampling operation is updated every iteration and also applies the computed deformation to subsurface vessel models and preoperatively designated fiducial positions by interpolating displacements from the deformed mesh.

For feature data points corresponding to a geometric model surface, the model-data error term becomes a sliding constraint taken to be the magnitude of the vector projection onto Sᵢ\hat{n}, the unit normal direction at the closest surface point:

f_{surface}=(Sᵢ\hat{n})^{T}pᵢ. Equation 23

This sliding constraint is maintained for centerline feature data points by taking the magnitude of the vector rejection of pᵢ from Sᵢ\hat{t}, the unit tangent vector at the closest point on the centerline model, which can be derived to be:

f_{centerline}=\sqrt{pᵢ^{T}pᵢ−(pᵢ^{T}Sᵢt)²}. Equation 24

Finally, the error term for single fiducial points is simply the Euclidean distance between the model-predicted and measured fiducial location:

f_{fiducial}=\sqrt{pᵢ^{T}pᵢ}. Equation 25

The energy penalty function is represented by the average strain energy density distributed over the mesh vertices:

$${f}_{E} = \frac{1}{2 M} {α}^{T} ( {J}_{ɛ}^{T} {J}_{σ} ) α \qquad Equation 26$$

where M is the number of nodes in the mesh. For all registrations, the weights in (Equation 20) for the falciform and inferior ridges are chosen to be 0.3 m⁻², the strain energy weight 10⁻⁸ Pa⁻², and all other weights are 1.0 m⁻². From an initial estimate β₀=0, Levenberg-Marquardt optimization iteratively solves for β by the step:

βₖ₊₁−βₖ=(J^{T}WJ+λdiag (J^{T}JW))⁻¹J^{T}W f Equation 27

where the minimized errors are f=[fᵢ, f_{E}], the square diagonal matrix W=diag(w_{F}/N_{F}, w_{E}) are function weights, the regularization parameter λ is controlled by a trust region prediction ratio, and the Jacobian of the error is J=∂f/∂β. The termination condition is established as |Ω(βₖ₊₁)−Ω(βₖ)|<10⁻¹².

### Experimental Creation of Sample Phantom for Deformation

The deformable hepatic phantom used constructed according to the present disclosure consisted of a combination of purified water, 1.5 wt % agar-agar powder (Thermo Fischer Scientific, Waltham, Ma.), and 50 wt % liquid egg whites (Break Free Liquid Egg Whites, The Kroger Company, Cincinnati, Ohio). Liquid egg whites can be used to produce a permanent visual history of the thermal induced ablation lesion, similar in nature to the ablation lesions which form in tissue. Egg whites contain around 10% ovalbumin protein dissolved in 90% water with nearly no carbohydrate or fat content. Thermal denaturing of the ovalbumin protein leads to aggregation which causes optical scattering and a large reduction in the T2-, relaxation coefficient of the material. The resulting ablation lesions can be imaged using T2-weighted MM (as shown in FIG. 14B) and visually observed with mock histology by sectioning the phantom along the midline of the ablation antenna and backlighting the section.

In some examples of the creation of a phantom, powdered agar-agar can be thoroughly mixed with water and heated to boiling on a hot plate while being continuously stirred to produce a 1.5% agar gel. The solution can then be cooled to <60° C. with continuous stirring before adding the liquid egg whites. This cooling ensures that no protein denatured prematurely. The mixture can then be mixed for 1 minute and then poured into the phantom mold to allow the gel to set. A plaster negative derived from contrast-enhanced CT imaging of a patient liver was used as the phantom mold to produce a hepatic phantom with clinically-relevant anatomical structure (as shown in FIG. 14A).

A phantom constructed according to this method can achieve relatively low rigid registration results as compared to prior phantom studies. Such results indicate that the degree of applied soft-tissue deformation is less than previously achieved in the art. This is in large part due to the nature of the agar-albumin phantom which is prone to shearing when subjected to large deformations. However, 20-30 mm of deformation is adequate for understanding the relative performance under assumptions of rigid versus non-rigid deformation-corrected alignment.

### Experimental Deformation of Sample Phantom

An exemplary phantom can be treated with 915 MHz microwave ablation to create a visible ablation lesion. The ablated phantom can be imaged using T2-weighted MRI, from which the phantom boundary, ablation zone, antenna tip, and antenna insertion point can be segmented in the initial “pre-deformation” pose of the phantom. Support blocks can be inserted beneath the phantom to change the underlying posterior support surface shape as well as to shift the ablation. The phantom can be then re-imaged in this “post-deformation” state, providing the same information as before but in this new deformed pose. This two-step process effectively provides all procedural delivery information and ablation physical outcomes in a ‘before’ and ‘after’ deforming event.

Image-to-physical registration can then be performed by registering the initial pre-deformation image to the mock operating room/interventional suite (OR/IS) surface digitization of the organ generated from the post-deformation imaging data (surgical/OR setting utilizing partial surface data, interventional/IS setting utilizing full surface data). This experimental setup allowed the ablation information to effectively be used as a geometric target for registration assessment. In addition, a retrospective model of the MWA procedure (i.e. a model of antenna power deposition and thermal distribution) can be simulated in the pre-deformation pose given the antenna location segmented from Mill. Together, these experiments show how well a computationally-modeled, deformation-corrected ablation prediction performs versus the ground truth ablation extent and location (i.e. the evaluation of a model-based therapeutic and localization system). The experiment included a total of 3 ablations present in 8 image-to-physical registration scenarios.

The proposed modeling and registration framework can be evaluated across a range of clinically-relevant organ deformations. With respect to open surgery, deformations occur due to the introduction of packing material beneath and around the organ following mobilization of the organ from surrounding anatomy. With respect to intervention, changes between diagnostic and intraprocedural presentation incur shape change. To impose soft tissue deformations in an experimental set-up, silicone support blocks (roughly 20-30 mm thick) can be inserted beneath varying areas of the phantom. FIG. 14A shows an exemplary extent of deformation induced in a phantom. In total, 4 unique applications of deformation were applied to the phantom to test different levels of intraprocedural deformations. In case 1, support material was placed beneath the lateral superior right lobe, raising the largest volume of the phantom. In case 2, support material was added beneath both the lateral superior and inferior right lobe, causing the right lobe to rise and rotate about the falciform ligament. In case 3, support material was inserted beneath the lateral inferior right lobe and the left lobe, causing the medial area of the liver to sag. Finally, in case 4 support material was inserted beneath the lateral inferior right lobe, causing it to rise.

### Experimental Collection Of Deformation Data

Five phantom deformation (i.e. 1 pre-deformation image set and 4 post-deformation image sets) 3D models can be generated from each set of MR images from an exemplary phantom using ITK-SNAP. Salient feature regions can be manually designated from the surface of each model. These image-to-physical registration methods can be clinically implemented using sparse digitization of the anterior organ surfaces attained intraoperatively.

Sparse anterior surface data, akin to what would be available clinically, can be generated experimentally by gathering sparse surface data from actual clinical cases and superposing the patterned data to a mock phantom image volume in its deformed state. That can be accomplished using a weighted salient feature registration method. Once initialized, an affine registration can be performed to account for any differences in organ size between the clinical and phantom data.

Next, the clinical surface data can be projected to their closest points on the deformed phantom surface, resulting in unique realistic sparse anterior surface designations for each case. However, no additional noise need be added to these synthesized sparse surface data. All synthesized designations can have an extent of organ coverage between 25-40%, which is within the range of typical clinical data acquisition. The above process can be performed in a reciprocal workflow allowing for a second set of novel conditions. In total, this creates 8 registration scenarios (the original 4 cases and the 4 reciprocal instances) for the results herein, each with an independent set of simulated physical space data to drive the registrations.

### Experimental Results: Deformation Data

Table I shows average TRE results from driving the registrations with full surface, sparse surface, and resampled sparse surface data as with respect to method 400.

TABLE I
Average and standard deviation target registation
error are presented for each source of surface data and
each evaluated method of registration.

| | Average Target Registration Error [mm] | |
|---|---|---|
| Surface | Rigid | Deformation |
| Data | registration | Correction |
| Full | 5.6 ± 2.3 | 2.5 ± 1.1 |
| Sparse | 6.0 ± 2.3 | 3.7 ± 1.4 |
| Resampled | 4.9 ± 2.1 | 3.8 ± 1.3 |

Table II shows average PPV results from driving the registrations with full surface, sparse surface, and resampled sparse surface data are presented for both methods of registration. Table II presents the maximum volumetric accuracy of the retrospective ablation modeling utilized within this study under the condition of perfect localization.

TABLE II
Average and standard deviation volumetric overlap
are presented as the positive predictive value for each
source of surface data and each evaluated method
of registration. Additionally, the positive predictive
value is presented for the case of perfect localization
to distinguish model error from registration error.

| | Average Positive Predictive Value [%] | |
|---|---|---|
| Surface | Rigid | Deformation |
| Data | registration | Correction |
| Full | 67.0 ± 11.8 | 85.6 ± 5.0 |
| Sparse | 64.8 ± 12.4 | 77.1 ± 8.0 |
| Resampled | 69 ± 11.1 | 75.1 ± 6.5 |

| | |
|---|---|
| Perfect | 96.3 ± 0.3 |
| Localization | |

In addition to reporting the antenna point target errors in Table I, the volumetric error associated with the retrospectively modeled ablation zones is shown in Table II. Again, these results demonstrate improved localization by the deformation-corrected method of the present disclosure. FIGS. 7A-7F visualize clearly better fidelity when comparing the overlap of the red ablation on rigid registration (FIGS. 7A-7B) to that of the overlap of the deformation corrected registration using sparse data (FIGS. 7C-7D) and finally complete surface data (FIGS. 7E-7F). As with the point target errors, for each data source (Table II) the deformation correction method significantly outperforms the rigid alignment method of (p<0.001).

Furthermore, the sparse data resampled results showed improvement to the rigid registration method. However, this was not the case for the deformation correction methods. This is largely because the simulated sparse surface data did not include digitization noise (i.e. the sparse surface data was selected directly from the MM surface) which the resampled method was specifically designed to reduce.

In summary, this application is a significant advancement in the field of hepatic image-guided ablation, as soft-tissue deformation is a considerable limitation to current rigid registration-based approaches. Conventional methods target accuracy of ablation antennas on the order of 5-10 mm, with the current best being a median accuracy of 4.2 mm. In comparison, a method of deformation correction according to the present disclosure can produce favorable accuracies of 2.5±1.1 mm and 3.7±1.4 mm when using full (IS) and sparse (OR) surface data, respectively.

Evaluation of the applied method thus shows significant improvement in localization accuracy when compared to a clinically-relevant rigid registration approach. Furthermore, incorporating a retrospective model of MWA into the navigational framework, provides an important evaluation of the interplay between localization accuracy and volumetric overlap of predicted and ground-truth ablation zones. While future work is necessary to apply this modeling and navigational framework as a prospective, targeting approach, the deformation correction method applied in the present disclosure is certainly an advancement toward improved localization in hepatic MWA procedures. Going further, this work creates a new concept of ‘model correction and feedback’ that includes a new biophysical domain, namely the deposition of ablative energy, and its corresponding thermal evolution. This combined mechanics/energy framework represents a first realization in a more comprehensive model-predictive paradigm for an important image-guided therapeutic process in use today.

### Experimental Creation of Sample Phantom With Fat Content

A heat-sensitive gel phantom can be constructed consisting of liquid egg whites, vegetable shortening, and the remainder with agar gel. Egg whites consist of approximately 90% water and 10% dissolved protein. The denaturing of ovalbumin protein within the egg whites provides a visualization of thermal damage within the phantom. Thermal denaturation of the protein causes aggregation, leading to optical scattering. This denaturation causes the thermal lesion to be clearly visible when prepared in mock histology. Vegetable shortening can be used to introduce a controllable variability to the thermal and electrical properties of the phantom. Table III below demonstrates the general reduction in thermal and electrical properties of vegetable shortening compared to the other phantom components. The ranges of fat included within this study were chosen to represent the clinical presentation of fatty liver disease (i.e. 5-10% of liver weight).

TABLE III
Dielectric and thermal properties of the agar-albumin-
fat phantom components as reported in the literature.

| | Agar-albumin Phantom | | |
|---|---|---|---|
| | Agar-water | Vegetable | Liquid Egg |
| | gel (1.5%) | Shortening | Whites |
| σ [S/m] | 0.05 - 0.4^{[34]} | 1.04 × 10^{-5 [38]} | 1^{[42]} |
| ε | 67 - 84^{[35]} | 2.53 - 2.665^{[39]} | 50.2^{[42]} |
| c [J/Kg-K] | 3900^{[36]} | 1670^{[40]} | 3414^{[43]} |
| k [W/m-K] | 0.5 - 0.55^{[37]} | 0.155 - 0.170^{[41]} | 0.522^{[43]} |

To make this agar-albumin-fat phantom, 1.5 wt % agar powder (Thermo Fisher Scientific, Waltham, Mass.) can be mixed with an appropriate volume of purified water. The solution can then be heated gradually until boiling on a hot plate while being continuously stirred. After the agar gel exceedes 60° C., the desired amount of vegetable shortening can be introduced (Crisco, The J.M. Smucker Company, Orrville, Ohio). Once heated, the solution can then be cooled below 55° C. with continuous stirring, at which point 50 wt % liquid egg white (Break Free Liquid Egg Whites, The Kroger Company, Cincinnati, Ohio) can be added and mixed thoroughly for 1 minute.

The mixture can then be poured into the phantom mold and begin to solidify once cooled below 35° C. Note that the liquid egg white solution must be added when the temperature of the agar gel is below 60° C. to avoid prematurely denaturing the ovalbumin protein.

A cubic acrylic box with a volume of 1 L can serve as both the phantom mold and enclosure during the ablation procedures. The lid to the enclosure can incorporate a series of holes, centered 5 mm apart, along the midline of the phantom to enforce consistent placement of the ablation antenna and, if present, temperature sensors. During each experiment, the ablation and temperature sensors can be positioned within the phantom using these guides and rigidly fixed in position at measured depths.

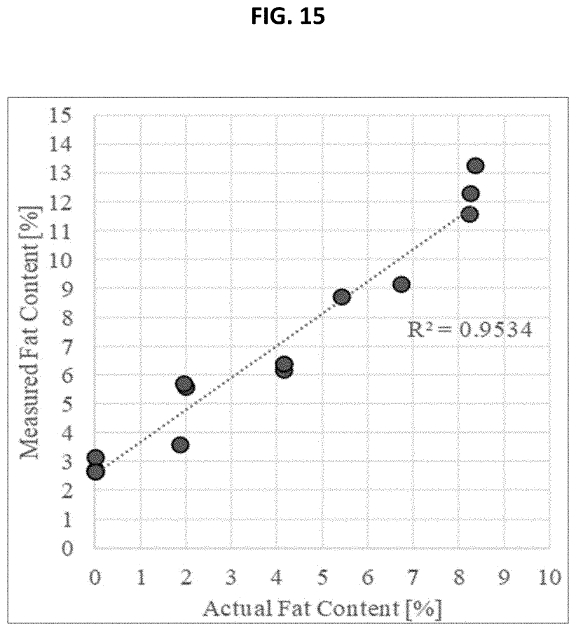

In total, 6 agar-albumin phantoms with no fat can be created for a model validation study (shown in FIGS. 10A-10F) and 15 agar-albumin-fat phantoms of varying fat percentages can be created for the phantom property model study (discussed with respect to FIG. 8).

### Experimental Data of Sample Phantom With Fat Content

When considering heating near the active region of the antenna, within the zone of the largest electromagnetic energy deposition, FIGS. 11A-11F shows very rapid heating within the first 5-7 minutes of the procedure. This rapid heating is often under predicted by the model before correcting by the final time point. Cases 3-4 in Table IV present the greatest rate of heating out of the 6 cases, and accordingly resulted in the reconstruction of the largest values of σ. Contrasting these results to the more gradual temperature profiles associated with the distant temperature sensors (shown in FIGS. 11A-11F), heating far from the ablation antenna can be shown to be dominated by heat transfer rather than electromagnetic energy deposition and that model-predicted temperatures throughout the procedure are more consistent (particularly for the farthest sensors from the active region of the ablation antenna).

TABLE IV
Optimized electrical and thermal conductivities
for each experimental case of the base agar-albumin
phantom in the model validation study.

| | Agar-albumin Phantom | |
|---|---|---|
| | Electrical | Thermal |
| Case # | conductivity (σ) | conductivity (k) |
| 1 | 0.57 | 0.67 |
| 2 | 0.44 | 0.34 |
| 3 | 0.58 | 0.62 |
| 4 | 0.58 | 0.67 |
| 5 | 0.54 | 0.47 |
| 6 | 0.50 | 0.62 |
| Average | 0.53 ± 0.05 | 0.56 ± 0.13 |

FIG. 15 provides additional insight into the show exemplary electrical and thermal conductivity data shown with regard to FIGS. 12A-12B. FIG. 15 shows measured fat content from MR versus actual fat for n=15 phantoms, where a baseline was not calibrated. With respect to mDIXON preliminary data used to quantify fat in FIG. 15, a heat-sensitive gel phantom was created consisting of liquid egg whites, vegetable shortening, and agar gel. Vegetable shortening was used to control fat content and consequently to mimic thermal and electrical property changes in a fatty liver. It should be noted that agar/albumin phantoms were carefully constructed to have thermal and electrical properties similar to reported liver properties (typically within 20% of reported values). Ranges of fat introduced were chosen to represent the clinical presentation of fatty liver disease (i.e. typically 5-10% of liver weight for NAFLD). MRI examination of each phantom was achieved with a 3T Intera Achieva MR scanner (Philips Healthcare, Netherlands). The commercial mDIXON Fat Quant imaging sequence was used to acquire fat fraction images of each of n=15 phantoms. The mDIXON Fat Quant protocol has a reported accuracy of ±3.5% and reproducibility of ±1.4%. FIG. 15 plots a notable relationship between mDIXON MR-measured fat (y-axis) and actual fat as estimated by component volume fractions (x-axis).

Within the literature, the influence of tissue perfusion has long been suggested as among the most significant properties affecting thermal ablation. As a result, the use of IVIM diffusion-weighted MR imaging as a means to assess perfusion was investigated. The conventional single-shot echo-planar imaging pulse sequence was used with diffusion gradients applied in three orthogonal directions with the following parameters: repetition time/echo time, 2700/63 msec; field of view, 14×14 cm; matrix, 128×128; slice thickness, 3 mm; number of signals acquired, eight; and multiple b values, 0, 50, 100, 150, 200, 400, 800, and 1000 sec/mm². For fat suppression, the Spectral Attenuated Inversion Recovery (SPAIR) technique was used to minimize the influences of field inhomogeneity. In this aim, series of controlled phantom experiments were performed to assess the predictive capability. With respect to analysis, diffusion-weighted imaging data is post-processed to extract the apparent diffusion coefficient (ADC), the true diffusion coefficient (D) associated with water diffusion, the pseudo-diffusion coefficient (D*) that is associated with perfusion-related incoherent microcirculation, and (f) the perfusion fraction. Similar experiences to previous studies that showed quite dramatic correlation between perfusion fraction and NAFLD disease extent in a rabbit model were anticipated.

Procedurally, ADC values are calculated by using all b values with a monoexponential fit with the equation: SI/SI₀=exp(−b*ADC),where SI₀ is the mean signal intensity of the region of interest for a b value of 0 sec/mm², and SI is the signal intensity for the higher b value. On the basis of the IVIM concept, the D,D*, and f values are calculated by using a nonlinear biexponential fit according to the following equation: SI/SI₀=(1−j)·exp(−b*D)+f·exp(−bD*). Four parametric maps of ADC, D, D*, and f are created.

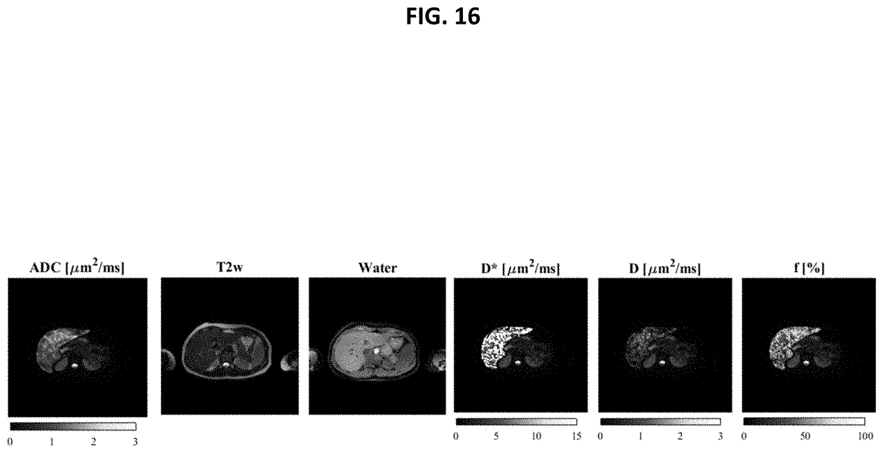

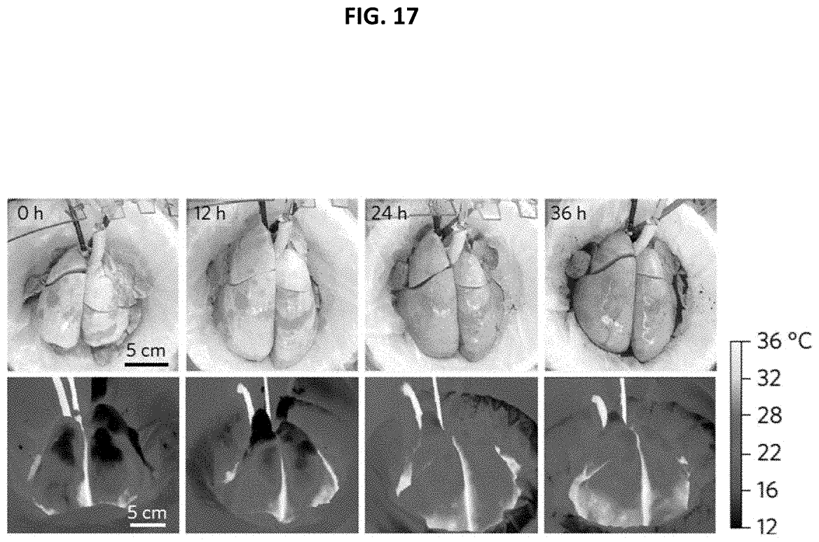

FIG. 16 shows exemplary intravoxel incoherent motion imaging for perfusion and diffusion of human liver tissue. Conventional imaging parameters and components associated with the bioexponential fit are presented with the critical perfusion fraction provided as f, a parameter to be used as an image-data-driven model property for perfusion. The images in FIG. 16, from left to right, show ADC, T2w, Water, D*, D, and f. Assuming that perfusion properties are determinable using these sequences, that will add an additional component to the model. Heat transfer and the resulting temperature history were solved using Pennes' bioheat equation:

$$ρ c \frac{\partial T}{\partial t} = ∇ \cdot κ ∇ T + Q - {Q}_{met} + {Q}_{p} \qquad Equation 28$$

where ρ [kg/m³] is mass density, c [J/kg·K] is specific heat capacity, κ [W/m·K] is thermal conductivity, T [K] is the tissue temperature, Q [W/m³] is heat generation due to absorbed electromagnetic energy, Qmet [W/m³] is metabolic heat generation, and Qp [W/m³] is heat loss due to perfusion. It is this last term that will take on a more direct form in light of IVIM imaging. More specifically:

Qₚ=η_{bl}c_{bl}ω(T_{α}−T)=m(T_{α}−T) Equation 29

where ρ_{bl} is the blood density, c_{bl} is the blood specific heat, co is the perfusion rate, and T_{α} is the arterial blood temperature (note, use of m is just as a lumped coefficient). Similar to how thermal conductivity became a linear function of fat content determined by fat quantification MR imaging, perfusion could be similarly determined from IVIM MR imaging and used to create patient-specific models for determining thermal dose in light of changes in perfusion.

In the experiments described above, physical measurements of ablations with analog scales and the relation of those measurements to cell death margins are determined with gross pathological and histological examination at the termination of the experiment (24 hours later to allow ablation lesion to set). The interim provides a window to acquire other intra-procedural data that may be valuable in monitoring MWA development or help with localization. Surface thermography (Fluke TIX580 60 HZ 60 Hz, 640×480, 0.93 mRad HD) and video data are provided continuously from the organ instrumentation system. In addition, periodically while the lesion is setting up, repeat tracked conventional B-mode ultrasound imaging is performed. Given that the proposed pre-clinical system is a rabbit and is significantly different in scale, a burr hole ultrasound transducer (Analogic Burr-Hole N11C5) is used for more realistic resolution and field of view when compared to human counterpart.

FIG. 17 shows time lapsed video images of organ thermographic data, in this case, of a lung. The data stream acquired includes time lapse video, and thermographic data over a 36-hour period of a porcine lung during organ perfusion recovery. The images, from left to right, show the porcine lung at 0 hours, 12 hours, 24 hours, and 36 hours. While the thermographic data is not as comprehensive as MR thermography per se, it is extensive and certainly is less cumbersome than the MR counterpart and potentially can be extended to the laparoscopic environment much easier. Other possibilities to assist could include ultrasound thermography as well.

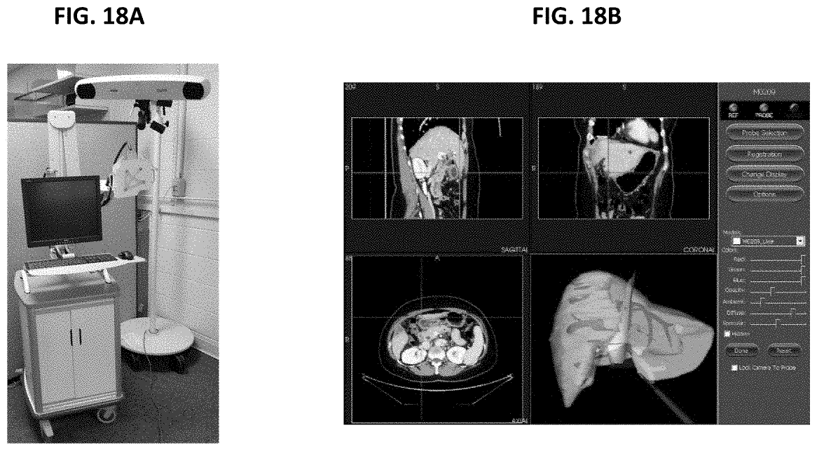

FIG. 18A shows an intraoperative guidance system to be used in accordance with an embodiment of the present disclosure, and FIG. 18B shows an intraoperative guidance display for the guidance system to be used in accordance with an embodiment of the present disclosure. The framework is comprised of two components: (1) a pre-processing package that allowed for segmenting the liver from contrast enhanced CT/MR data, and (2) an intraoperative guidance system and display which is shown in FIGS. 18A and 18B. With respect to segmentation, the approach in FIG. 18A is a semi-automatic level-set methodology and can segment an entire human liver in approximately 5-10 minutes. The platform in FIG. 18B is capable of recording data from a tracked stylus, laser range scanner, and ultrasound. Lastly, it should be noted that a prototype image-to-physical nonrigid registration approach using sparse surface data of the liver within the open surgery context has been used. New developments using additional sparse subsurface data, i.e. sparse vessel contours, may provide even further accuracy improvements.

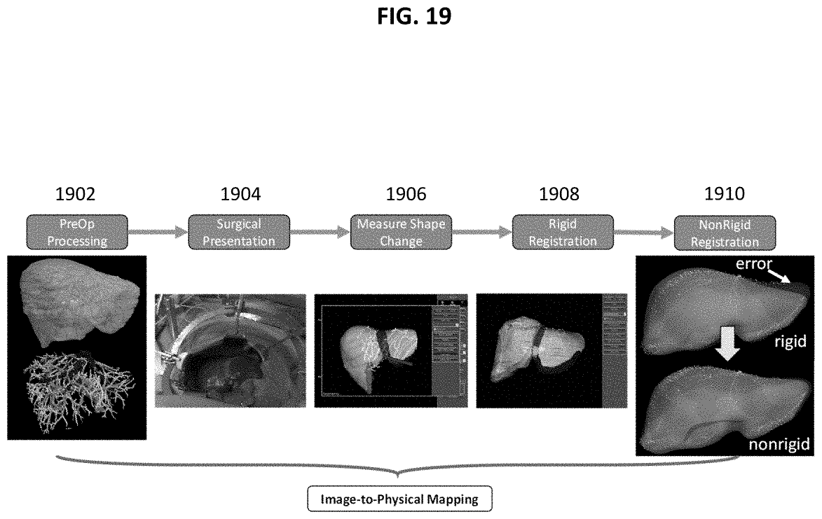

FIG. 19 shows a model-based process for registration process overview, in accordance with an embodiment of the present disclosure. That process overview as shown is a case of sparse data taken from the anterior liver surface by swabbing the liver surface with an optically tracked stylus. Results to date have been very promising, as evidenced by the reduction of error from rigid registration to our nonrigid method in right panel of FIG. 19. At step 1902, “PreOp Processing,” the process commences with pre-operative processing and imaging to map the organ. At step 1904, “Surgical Presentation,” the processed images are used in conjunction with a surgical procedure. At steps 1906, “Measure Shape Change,” and 1908, “Rigid Registration,” a robust developmental framework using mock soft-tissue phantoms is created involving a template design process for effective phantom-to-human translation. Given the considerable difference between the human liver and the rabbit liver, the registration framework will need to be customized for rabbit organ geometry. For example, with respect to registration, the inferior ridges of the lateral left lobe, medial left lobe, and quadrate lobe will be used as salient features, and quite possibly the caudate which is different than the clinical counterparts (e.g. red, blue, and green regions in FIG. 19, below steps 1906 and 1908). In addition, the control surfaces for applied nonrigid deformations will have to be adjusted to reflect the open rabbit configuration. At step 1910, “NonRigid Registration,” to give some sense of performance of the framework, at human length scales with a complete anterior liver surface, target registration errors on average throughout a mock human liver were approximately 2.4+/−1.3 mm. In the in vivo human counterpart over a limited number of cases (n=6) that used subsurface vessels for target validation, the average target error was 2.7+/−0.7 mm, which is quite consistent. With that, assuming a simple scale reduction in error based on a reduced lever-arm effect, an average target error of ˜1 mm is the objective.

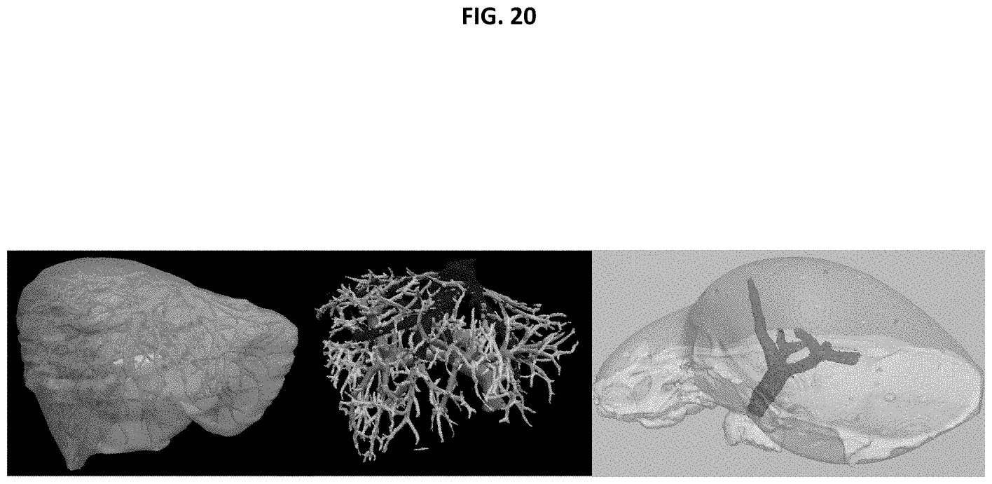

Another important realization is concerned with major vasculature effects on thermal dose deposition as well as being used to assist in image-to-physical registration. To capture these effects for model use and registration enhancement, GD-DTPA Magnevist could be used to create vascular maps of the liver. FIG. 20 shows an exemplary reproduction of these maps. To handle within the model controller, specific structures of the vasculature can be built within the computer model. With respect to the electromagnetic effects, these can be modeled with interior elements and assigned properties associated with blood. With respect to thermal effects, these can be assigned a fixed body temperature.

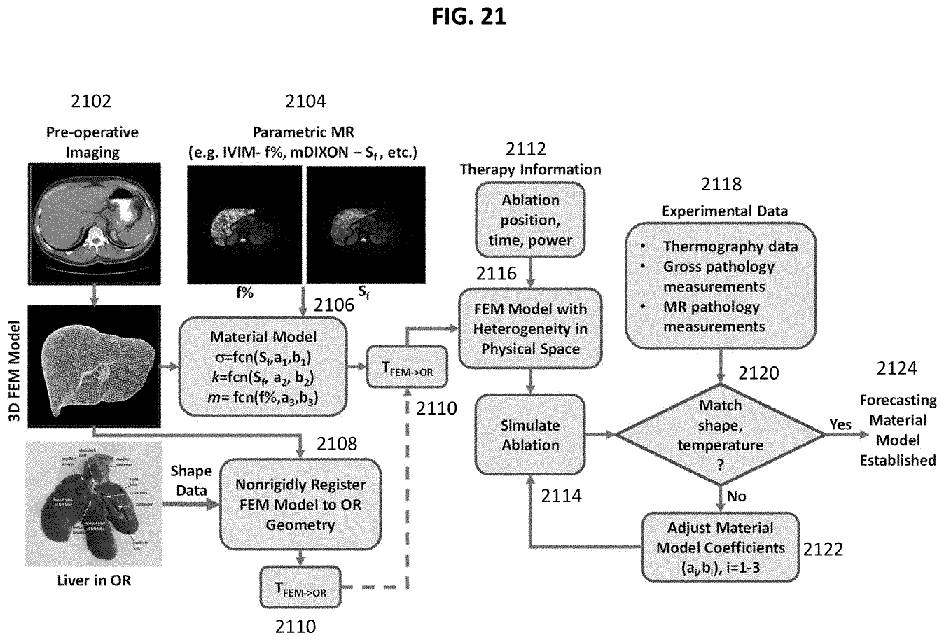

Going further with this realization, the basic premise is that a material model that relates MR-imaging quantities (fat quantification MR and IVIM MR) to model properties is possible and that will lead to better forecasting of therapy outcome via the model-based controller, in this case thermal ablation therapy. To achieve this, material models are needed. FIG. 21 shows a method to establish an image-driven forecasting material model in accordance with an embodiment of the present disclosure. The general approach begins with the acquisition of quantitative MR scans (e.g. mDIXON, IVIM, etc.). FIG. 21 commences with step 2102, “Pre-operative Imaging,” where processed images are provided, as has been described using the techniques above. At step 2104, “Parametric MR,” imaging data is acquired that characterizes the tissue. A materials model is applied to calculate the electrical conductivity (e.g. dielectric properties), σ, the thermal conductivity (e.g. thermal properties), k, and the lumped perfusion coefficient (e.g. perfusion properties and/or vascular properties), m, at “Material Model,” step 2106. These materials models are all based on respective measures from MR indicated as S_{f} and f % produced from step 2104 and the regression coefficients that make up each model. For example:

σ=a₁S_{f}+b₁, k=a₂S_{f}+b₂, m=a₃(f%)+b₃

would be a linear form where the respective properties represent simple constant or monotonic relationships between property and signal, where a represents the slope of the linear function and b represents the intercept. Those equations are just one possible functional relationship. It is possible that properties may need to be represented at bivariate models dependent on both or higher order functions or other nonlinear functions (e.g. exponential, etc.). Mass transport properties may also be incorporated in the calculation of the material model. With the functional form established but not tuned, the right side of FIG. 21 demonstrates one realization of fitting/tuning a material model. The material model contemplated may be comprised of magnetic resonance imaging methods that quantify fat and perfusion, recorded ablation settings, recorded temperature data, direct pathological measurements, and a post-ablation follow-up with imaging. As an example, the basic concept is to have a body of data that has information regarding fat quantification MR, and IVIM MR, recorded ablation settings, and a post-ablation follow-up with imaging. Using this data, a prospective relationship as shown in equations in step 2106 is determined in the fitting process and subsequently used to computationally forecast therapeutic delivery for planning and interventional purposes. Other measurements could be used as shown in FIG. 21. At step 2108, shape data from an organ and 3D FEM model data is collected and used to nonrigidly register the Finite Element Model (FEM) Model to the shape of the organ as presented during surgery or intervention . At step 2110, using shape data acquired during MWA intervention, the model is deformed to match the procedural presentation using the nonrigid methods described herein (T_{FEM→OR}).

Then, at step 2112, “Therapy Information,” using the data collected during the procedure, i.e. ablation shape from gross pathology, precise antenna locations from tracking, MW generator power settings, ablation times, B-mode ultrasound, and thermographic spatial surface data, comparisons between simulated ablations 2114 and the measurement counterparts will be performed. Assuming an initial functional relationship that relates signal to material parameter, e.g. perhaps linear (in FIG. 21, these are the parameters aᵢ,bᵢ, i=1-3), a 3D heterogeneous material property finite element model is generated 2116. At step 2118, “Experimental Data,” thermography data, gross pathology measurements, MR pathology measurements, and other similar data is collected. At step 2120, that data is used to determine whether the simulated ablation 2114 results in a proper fit to, for example, the ablation lesion shape and temperature. If different, parameters associated with the material model will be iteratively adjusted 2122 (aᵢ,bᵢ, i=1-3 in FIG. 21) until a proper fit is achieved. Once there is a match of shape and temperature against the model, at step 2124, “Forecasting Material Model Established,” the model is determined to have a proper fit and is applied in the procedure. The procedures contemplated include, but are not limited to, tissue ablation, neurostimulation, neuromodulation, convection therapy, and/or diffusion-delivered therapy.

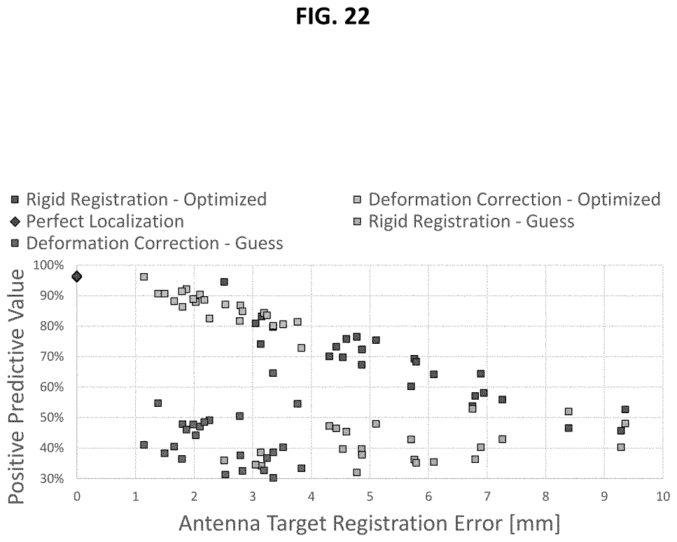

FIG. 22 shows a graph with predicted ablations under conditions of inaccuracies with respect to localization and under conditions where material properties for MWA forecasting were based on volume-weighted component models. In addition, results are also shown when deformations are taken into account, and when material properties are optimized. The result reflects an effort to understand the significance of incorrect property estimates relative to localization error within the context of predicting ablation lesions. Interestingly, any benefit in localization gained from accounting for deformation is lost when the ablation properties are incorrect.

At its simplest level, the overarching hypothesis is that a consistent model can be identified, and this subject-specific model will be significantly better at dose prediction than the standard-of-care power-time-size ablation tables provided by industry. The only prohibitive step clinically is the gross pathology measurement of the lesion. This would have to be substituted with post-operative imaging. In the protocol, post-ablation MR imaging is collected prior to euthanizing and pathology, so this will be investigated. For the purposes of the material model though, ground truth gross pathology is utilized.

The terminology used herein is for the purpose of describing particular examples only and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art to which this invention belongs. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

While various examples of the present invention have been described above, it should be understood that they have been presented by way of example only, and not limitation. Numerous changes to the disclosed examples can be made in accordance with the disclosure herein without departing from the spirit or scope of the invention. Thus, the breadth and scope of the present invention should not be limited by any of the above described examples. Rather, the scope of the invention should be defined in accordance with the following claims and their equivalents.

1. A computer-implemented method comprising:
collecting pre-operative imaging data and shape data associated with a patient tissue;
calculating a material model comprised of dielectric, thermal, mass transport, perfusion, and/or vascular properties;
fitting the material model to previously collected experimental data associated with the patient tissue; and
generating therapy guidance based on the material model and the shape data for a medical procedure.

2. The computer-implemented method of claim 1, wherein the medical procedure is one of tissue ablation, neurostimulation, neuromodulation, convection therapy, and/or diffusion-delivered therapy.

3. The computer-implemented method of claim 2, wherein the tissue ablation is performed by radiofrequency, microwave, cryoablation, high-frequency ultrasound, or irreversible electroporation.

4. The computer-implemented method of claim 1, further comprising deforming the material model using nonrigid methods.

5. The computer-implemented method of claim 1, further comprising simulating ablation therapies on the patient tissue to verify the fit of the material model.

6. The computer-implemented method of claim 4, wherein the simulated ablation therapies are compared to the previously collected experimental data, which is comprised of thermography data or other temperature measurement data, gross pathology measurements, and MR pathology measurements associated with the patient tissue.

7. The computer-implemented method of claim 1, wherein the material model calculation is comprised of imaging methods that quantify fat and perfusion, recorded ablation settings, recorded temperature data, direct pathological measurements, and a post-ablation follow-up with imaging.

8. A non-transitory computer-readable medium having stored thereon computer-readable instructions for:
collecting pre-operative imaging data and shape data associated with a patient tissue;
calculating a material model comprised of dielectric, thermal, mass transport, perfusion, and/or vascular properties;
fitting the material model to previously collected experimental data associated with the patient tissue; and
generating therapy guidance based on the material model and the shape data for a medical procedure.

9. The non-transitory computer-readable medium of claim 8, wherein the medical procedure is one of tissue ablation, neurostimulation, neuromodulation, convection therapy, and/or diffusion-delivered therapy.

10. The non-transitory computer-readable medium of claim 9, wherein the tissue ablation is performed by radiofrequency, microwave, cryoablation, high-frequency ultrasound, or irreversible electroporation.

11. The non-transitory computer-readable medium of claim 8, further comprising deforming the material model using nonrigid methods.

12. The non-transitory computer-readable medium of claim 8, further comprising simulating ablation therapies on the patient tissue to verify the fit of the material model.

13. The non-transitory computer-readable medium of claim 12, wherein the simulated ablation therapies are compared to the previously collected experimental data, which is comprised of thermography data or other temperature measurement data, gross pathology measurements, and MR pathology measurements associated with the patient tissue.

14. The non-transitory computer-readable medium of claim 8, wherein the material model calculation is comprised of imaging methods that quantify fat and perfusion, recorded ablation settings, recorded temperature data, direct pathological measurements, and a post-ablation follow-up with imaging.

15. A computer-implemented method comprising:
obtaining spatially encoded information about patient tissues of interest during a medical procedure;
modeling a tissue response for the patient tissues of interest associated with the medical procedure; and
integrating the spatially encoded information and the issue response to generate guidance during the medical procedure.

16. The computer-implemented method of claim 15, wherein the medical procedure comprises an ablation procedure using an ablation probe.

17. The computer-implemented method of claim 16, wherein the tissue response comprises at least one of tissue deformation to correct for a location of the ablation probe, an electric field distribution generated from the ablation probe during the ablation procedure, a thermal energy propagation in the patient tissues of interest due to molecular agitation from an electric field generated during the ablation procedure, and/or an estimate of tissue damage as a result of heating during the ablation procedure.

18. The computer-implemented method of claim 17, wherein tissue response provided by the deformation, or electric field distribution, the thermal energy propagation, and/or the estimate of tissue damage can be compared to spatially encoded information to guide the medical procedure.

19. The computer-implemented method of claim 16, wherein the guidance comprises at least one of a location of the ablation probe or a prediction of outcome for the patient tissues of interest in response to the ablation procedure and in reference to spatially encoded information.

20. The computer-implemented method of claim 15, wherein the ablation procedure is a microwave ablation procedure.
